(19) United States
(12) Patent Application Publication
(10) Pub. No.: US 2024/0415666 A1
(43) Pub. Date: Dec. 19, 2024

(54) ORTHOPEDIC SURGICAL INSTRUMENTS AND METHODS OF USE

(71) Applicant: Foundation Surgical Group, Inc., Scottsdale, AZ (US)

(72) Inventors: Randal R. Betz, Bradenton, FL (US); Michael Sherman, Memphis, TN (US); Charlie Barfield, Hernando, MS (US); Dimitri K. Protopsaltis, Memphis, TN (US)

(73) Assignee: Foundation Surgical Group, Inc., Scottsdale, AZ (US)

(21) Appl. No.: 18/813,627

(22) Filed: Aug. 23, 2024

Related U.S. Application Data
(63) Continuation of application No. 18/736,212, filed on Jun. 6, 2024.
(63) Continuation-in-part of application No. 18/424,843, filed on Jan. 28, 2024.
(63) Continuation-in-part of application No. 18/328,876, filed on Jun. 5, 2023.
(63) Continuation-in-part of application No. PCT/US2023/067912, filed on Jun. 5, 2023.
Application No. 18/736,212 is a continuation of application No. 18/424,843, filed on Jan. 28, 2024.
Application No. 18/424,843 is a continuation of application No. 18/328,876, filed on Jun. 5, 2023.
Application No. 18/424,843 is a continuation-in-part of application No. PCT/US2023/067912, filed on Jun. 5, 2023.
(60) Provisional application No. 63/478,620, filed on Jan. 5, 2023.
(60) Provisional application No. 63/369,330, filed on Jul. 25, 2022.
(60) Provisional application No. 63/349,136, filed on Jun. 5, 2022.
(60) Provisional application No. 63/478,620, filed on Jan. 5, 2023.
(60) Provisional application No. 63/369,330, filed on Jul. 25, 2022.
(60) Provisional application No. 63/349,136, filed on Jun. 5, 2022.

(51) Int. Cl. A61F 2/44; A61F 2/30

(52) U.S. Cl. CPC A61F 2/4425; A61F 2002/30405; A61F 2002/443

(57) ABSTRACT

An elevator tool for preparing a surgical site for an implant device is disclosed. The elevator tool comprises a distal paddle coupled to a paddle handle assembly and a positioning grip. The paddle handle assembly influences movement of the distal paddle and the positioning grip influences position of the distal paddle. The paddle handle assembly is coupled to the distal paddle with an inner shaft extending through the receiving grip whereby the distal paddle can be placed on the contralateral side of a bone and can be moved by the paddle handle assembly oriented on the ipsilateral side of the bone. The distal paddle comprises an elongated member having at least one arm configured to be rotated at least about 90 degrees by the paddle handle assembly to remove obstructions on the contralateral side of the vertebral body from a position oriented on the ipsilateral side of the vertebral body.

### CROSS-REFERENCE TO RELATED APPLICATIONS

This application is a continuation in part application of U.S. patent application Ser. No. 18/736,212 filed on Jun. 6, 2024; this application is also a continuation in part application of U.S. patent application Ser. No. 18/424,843 filed on Jan. 28, 2024; this application is also a continuation in part application of U.S. patent application Ser. No. 18/328,876 filed on Jun. 5, 2023; this application is also a continuation in part application of PCT Pat. App. No. PCT/US2023/067912 filed on Jun. 5, 2023; U.S. patent application Ser. No. 18/736,212 is a continuation of U.S. patent application Ser. No. 18/424,843; U.S. patent application Ser. No. 18/424,843 is also a continuation application of U.S. patent application Ser. No. 18/328,876; U.S. patent application Ser. No. 18/328,876 claims benefit of U.S. Pat. App. No. 63/478,620 filed on Jan. 5, 2023; U.S. patent application Ser. No. 18/328,876 also claims benefit of U.S. Pat. App. No. 63/369,330 filed on Jul. 25, 2022; U.S. patent application Ser. No. 18/328,876 also claims benefit of U.S. Pat. App. No. 63/349,136 filed on Jun. 5, 2022; U.S. patent application Ser. No. 18/424,843 is also a continuation in part application of PCT Pat. App. No. PCT/US2023/067912 filed on Jun. 5, 2023; PCT Pat. App. No. PCT/US2023/067912 claims benefit of U.S. Pat. App. No. 63/478,620; PCT Pat. App. No. PCT/US2023/067912 also claims benefit of U.S. Pat. App. No. 63/369,330; PCT Pat. App. No. PCT/US2023/067912 also claims benefit of U.S. Pat. App. No. 63/349,136; and the entire contents of all are incorporated herein by reference.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

### 1. Field of the Invention

The present disclosure relates generally to surgical instruments and their use with orthopedic implant devices. The instruments may be used with spinal implant devices configured to augment the vertebral body or fuse multiple vertebral bodies to decompress neural elements and alter the alignment of the spine. The surgical instruments may also be used with orthopedic implant devices configured for use within other bones or other joints.

### 2. Background

During surgical operations, it is beneficial to minimize trauma to the patient's body. With a reduced level of trauma, particularly to muscles and nerves, patients are able to recover more quickly. Minimizing trauma is a benefit of minimally invasive surgical procedures as well as traditional open surgical procedures. For example, for minimally invasive surgery, the procedures typically have small incisions and use instruments and implants that can be inserted and manipulated through those incisions. Even in traditional open surgical procedures, there is a benefit to minimizing the incisions by using procedures such as those that can implant and manipulate devices from one side of the patient.

For example, for minimally invasive spinal (MIS) surgery, there are significant benefits to having devices and tools that can be inserted, manipulated, and implanted through a single small incision. Open surgical procedures can benefit from the same features. Additionally, MIS and open surgical procedures can benefit from specific devices and methods that can be used at particular angles relative to the implantation site. Accessing the implantation site from certain angles can minimize trauma, reduce the complexity of the surgery, and enable certain implant features, such as spinal correction angles, to be provided.

Examples of suitable devices and methods for such procedures include those disclosed in U.S. Pat. No. 11,259,936 issued on Mar. 1, 2022, pending U.S. patent application Ser. No. 18/424,843 filed on Jan. 28, 2024 and published as US2024/0164909 on May 23, 2024 and U.S. patent application Ser. No. 18/736,212 filed on Jun. 6, 2024; the entire contents of all are herein incorporated by reference.

Therefore, there is a need for instruments that assist in surgical procedures that minimize trauma to the patient by accessing the surgical site from particular angles and allow manipulation and securing of an implant device from that access location and trajectory.

### BRIEF SUMMARY OF THE INVENTION

The following summary is included only to introduce some concepts discussed in the Detailed Description below. This summary is not comprehensive and is not intended to delineate the scope of protectable subject matter, which is set forth by the claims presented at the end.

Within this description, the terms far, distal, and contralateral are used interchangeably and are intended to be interpreted as defining that one thing is distant from another such as distance from a point of origin, situated away from a point of origin, and pertaining to the other side. Also, the terms near, proximal and ipsilateral are used interchangeably within this description and are intended to be interpreted as defining a short distance away from another such as away from a point of origin, situated toward a point of origin and belonging to or occurring on the same side of a body.

Within this description and the descriptions of the patent applications to which this application claims priority to, the terms anchor frame and vertical member are used interchangeably to refer to the same structural element and are intended to be interpreted as any type of structural element having any position or angle relative to the cage to help secure implant system components to bone and as further described below.

Generally, orthopedic surgical instruments, implant systems and methods are disclosed for use with orthopedic implant systems.

In some embodiments of the surgical implements, implant systems and methods, the implant system is positioned and manipulated with instruments from one side of the bone. The ability to implant and manipulate the implant device from one side of the implantation site also provides the ability for the implant to be implanted from orientations that take advantage of the surgical benefits of approach orientations such as posterior, anterior, lateral or oblique.

To install the implant device, an insertion handle assembly is disclosed. The insertion handle assembly is generally configured to securely deliver the implant device to the implant location and assist in allowing other tools to manipulate the implant device so that it can be secured to the bone.

To prepare the bone for receiving the implant device, particularly to prepare the far-side of the bone for receiving a distal anchor or staple, an elevator tool is disclosed. The elevator tool is generally a far-side elevator tool configured to be positioned near the implant location and manipulated to clear or elevate obstructions from the far-side surface of the bone.

In one aspect, the present disclosure provides an elevator tool for preparing a surgical site for an implant device, the elevator tool comprising: a paddle handle assembly; a distal paddle; the paddle handle assembly operably coupled to the distal paddle whereby the paddle handle assembly influences a movement of the distal paddle; and a positioning grip extending between the paddle handle assembly and the distal paddle whereby the positioning grip influences a position of the distal paddle.

In some embodiments, the positioning grip comprises an outer sleeve having a receiving portion; and the paddle handle assembly is coupled to the distal paddle with an inner shaft extending through the receiving portion of the outer sleeve.

In some embodiment, the outer sleeve is coupled to a shell having a receiving portion; and the inner shaft extending through the receiving portion of the shell.

In some embodiments, the receiving portion of the outer sleeve is configured to allow for an extension and a retraction of the inner shaft in and out of the receiving portion of the outer sleeve; the outer sleeve having an outer sleeve stop; and the inner shaft has a measuring portion exposed outside of the receiving portion of the outer sleeve whereby when the outer sleeve stop is positioned against an ipsilateral side of a bone, the measuring portion identifies a distance from the ipsilateral side of the bone and the distal paddle.

In some embodiments, the elevator tool further comprises: the paddle handle assembly coupled to the distal paddle with an inner shaft; a lock coupled to the positioning grip; the paddle handle assembly comprising a handle; and the lock configured to engage the handle whereby when the lock is engaged with the paddle handle, the handle and the distal paddle are maintained in a locked position relative to the positioning grip.

In some embodiments, the distal paddle comprises an elongated member having at least one arm extending from a rotational axis; and the at least one arm is configured to be rotated at least about 90 degrees by the paddle handle assembly.

In some embodiments, the distal paddle comprises an elongated member having at least one arm extending from a rotational axis; and the at least one arm is configured to be rotated at least about 180 degrees by the paddle handle assembly.

In some embodiments, the distal paddle comprises an elongated member having at least one arm extending from a rotational axis; and the at least one arm is configured to be rotated unconstrained at least about 360 degrees by the paddle handle assembly.

In some embodiments, the positioning grip comprises an outer sleeve having a receiving portion; the outer sleeve coupled to a shell; the shell having a receiving portion; the paddle handle assembly is coupled to the distal paddle with an inner shaft extending through the receiving portion of the outer sleeve and the receiving portion of the shell; the paddle handle assembly comprises a handle coupled to the distal paddle with an inner shaft; the elevator tool further comprises a lock coupled to the positioning grip; the lock configured to engage the handle whereby when the lock is engaged with the handle, the handle and the distal paddle are maintained in a locked position relative to the positioning grip; the distal paddle comprises an elongated member having at least two arms extending from a rotational axis; and the at least two arms are configured to be rotated at least about 180 degrees by the handle whereby the at least two arms are configured to remove obstructions on a contralateral side of a vertebral body by manipulating the handle from a position oriented on the ipsilateral side of to the vertebral body.

In another aspect, the present disclosure provides an elevator tool for use in preparing a contralateral side of a vertebral body for an orthopedic implant, the elevator tool comprising: a paddle handle assembly; a distal paddle; the paddle handle assembly operably coupled to the distal paddle whereby the paddle handle assembly influences a movement of the distal paddle; a positioning grip extending between the paddle handle assembly and the distal paddle whereby the positioning grip influences a position of the distal paddle; the positioning grip comprises an outer sleeve having a receiving portion; the outer sleeve coupled to a shell having a receiving portion; the paddle handle assembly is coupled to the distal paddle with an inner shaft extending through the receiving portion of the outer sleeve and the receiving portion of the shell; the paddle handle assembly comprises a handle coupled to the distal paddle with an inner shaft; the distal paddle comprises an elongated member having at least one arm extending from a rotational axis; and the at least one arm is configured to be rotated by the handle whereby the at least one arm is configured to remove obstructions on the contralateral side of the vertebral body by manipulating the paddle handle from a position oriented on the ipsilateral side of the vertebral body.

In some embodiments, the elevator tool further comprises a lock coupled to the positioning grip; and the lock is configured to engage the handle whereby when the lock is engaged with the handle, the handle and the distal paddle are maintained in a locked position relative to the positioning grip and when the lock is not engaged with the handle, the handle and the distal paddle are able to rotate within the receiving portion of the outer sleeve.

In some embodiments, the at least one arm is configured to be rotated through an unconstrained 360 degree rotation when the lock is not engaged with the handle.

In another aspect, the present disclosure provides a method for preparing a contralateral side of an insertion site of a bone for an orthopedic implant, the method comprising: providing access to a bone; providing an elevator tool comprising a distal paddle, a paddle handle assembly and a shell; the distal paddle operably coupled to the paddle handle assembly; positioning, from a proximal side of a bone, the shell in an insertion site whereby the distal paddle is positioned on a contralateral side of the bone; and engaging the paddle handle assembly to move the distal paddle on the contralateral side of the bone to prepare the contralateral side of the bone for the orthopedic implant.

In some embodiments, the step of engaging the paddle handle assembly to move the distal paddle on the opposite side of the bone clears an obstruction on the contralateral side of the bone.

In some embodiments, the step of engaging the paddle handle assembly to move the distal paddle on the contralateral side of the bone elevates a tissue on the contralateral side of the bone.

In some embodiments, the step of engaging the paddle handle assembly to move the distal paddle on the contralateral side of the bone comprises rotating the paddle handle assembly to rotate the distal paddle to clear an obstruction or elevate a tissue on the contralateral side of the bone.

In some embodiments, the paddle handle assembly is operably coupled to the distal paddle with an inner shaft rigidly coupling the paddle handle assembly to the distal paddle; and the step of engaging the paddle handle assembly to move the distal paddle on the contralateral side of the bone comprises rotating the paddle handle assembly to rotate the inner shaft and the distal paddle whereby the distal paddle rotates to clear an obstruction or elevate a tissue on the contralateral side of the bone.

In some embodiments, the bone comprises a vertebral body and the contralateral side of the vertebral body comprises a contralateral sidewall of the vertebral body.

In some embodiments, the bone comprises a first vertebral body and a second vertebral body and the contralateral side of the bone comprises a contralateral sidewall of the first vertebral body and a contralateral sidewall of the second vertebral body.

In some embodiments, the paddle handle assembly is operably coupled to the distal paddle with a trigger mechanism and the step of engaging the paddle handle assembly to move the distal paddle on the opposite side of the bone comprises engaging the trigger mechanism to rotate the distal paddle whereby the distal paddle rotates to clear an obstruction or elevate a tissue on the opposite side of the bone.

In some embodiments, the step of engaging the paddle handle assembly to move the distal paddle on the contralateral side of the bone comprises engaging the paddle handle assembly to move the distal paddle in about 180 degrees of rotation on the contralateral side of the bone.

In some embodiments, the step of engaging the paddle handle assembly to move the distal paddle on the contralateral side of the bone comprises engaging the paddle handle assembly to move the distal paddle in an unconstrained 360 degrees of rotation on the contralateral side of the bone.

In some embodiments, the bone comprises one or more vertebra and the step of providing access to the one or more vertebra is from an anterior approach relative to the one or more vertebra.

In another aspect, the present disclosure provides an insertion handle assembly having a distal end configured to engage the proximal end of a cage of an implant device whereby the insertion handle assembly can be used to manipulate the implant device. In some embodiments, the insertion handle assembly comprises a switch and a handle grip wherein the handle grip is coupled to a cannulated inner sleeve. In some embodiments, the cannulated inner sleeve is received within a handle outer sleeve that is telescopically coupled to the handle grip. In some embodiments, the switch, the handle outer sleeve, the handle grip, and the cannulated inner sleeve are coupled whereby when the switch is rotated in one direction relative to the handle grip, the handle outer sleeve is translationally locked and cannot be pulled retrograde towards handle grip. In some embodiments, when the switch is rotated in the other direction, the handle outer sleeve is free to translate and retract relative to cannulated inner sleeve and handle grip.

### Intravertebral Applications

Intravertebral use of the surgical instruments and implant systems is intended to restore foraminal height and treat vertebral body wedging, which result from microfractures and collapse of the vertebral body endplates. Correction of these deformities in the vertebral body via osteotomy and placement of the vertebral implant will reduce the back and leg pain by realigning the facet joints and opening the foramen in this select group of patients.

### Intervertebral Applications

Intervertebral use of the disclosed surgical instruments and implant systems is intended to fuse opposing vertebral bodies to eliminate painful motion and/or to restore anatomic alignment, height and stability to the spine following a spinal decompression. This fusion eliminates motion between vertebrae and also prevents the irritation and stretching of nerves and surrounding ligaments and muscles. Intervertebral use of the implant system generally provides an implant that is able to be secured to the inferior and superior endplates of two opposing vertebrae to facilitate a fusion.

### Applications with Other Joints

Surgical instruments and implant devices similar in design to the disclosed instruments and implant systems may be used as an arthrodesis implant device in an arthrodesis procedure for other joints. As done for the joining of two vertebrae, an implant device may be provided that is configured to be secured to opposing sides of adjoining bones in a joint to fuse those bones.

Other objects, features, and advantages of the systems and techniques disclosed in this specification will become more apparent from the following detailed description of embodiments in conjunction with the accompanying drawings.

### BRIEF DESCRIPTION OF THE SEVERAL VIEWS OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A shows the sagittal, coronal, and transverse planes of the human body;

FIG. 1B illustrates the different axis, orientations and placement approaches used with an example of an implant systems;

FIG. 1C illustrates an example osteotomy of the vertebral body when viewed in the coronal plane;

FIG. 2A shows example implant devices implanted as intervertebral and intravertebral implants;

FIG. 2B shows an anterior view of an example implant device implanted as an intervertebral implant;

FIG. 3 shows a perspective view of an example embodiment of an implant system;

FIGS. 4A-4F show different views of an example of an insertion handle assembly where FIG. 4A shows the insertion handle assembly separated from an implant device, FIG. 4B shows a close-up view of the coupling elements of the insertion handle assembly with the implant device, FIG. 4C shows the insertion handle assembly coupled to the implant device and FIG. 4D shows a close-up view of the coupling of the insertion handle assembly and the implant device, FIG. 4E shows details of the distal end of the insertion handle assembly, and FIG. 4F shows details of guide and lock mechanisms of the insertion handle assembly;

FIGS. 5A and 5B show elements of an example of the staple drive handle assembly where FIG. 5A shows the engagement of the staple drive handle with the staple shaft and nut and FIG. 5B shows the distal end of the staple drive handle assembly;

FIGS. 6A and 6B show different views of an example of a far-side elevator tool where FIG. 6A shows the paddle in an insertion alignment and FIG. 6B shows the paddle in a deployed alignment;

FIGS. 7A-7E show different views of an example of a far-side elevator tool where FIG. 7A shows a paddle handle in an insertion alignment, FIG. 7B shows the paddle handle in an extended position, FIG. 7C shows the paddle handle rotating and FIGS. 7D and 7E show the measuring portion exposed;

FIGS. 8A-8E show different views of another example of a far-side elevator tool where FIG. 8A shows the far-side elevator tool in an insertion alignment, FIG. 8B shows a close up view of the distal end of the far-side elevator tool with the distal paddle in an insertion alignment, FIG. 8C shows a close up view of the proximal end of the far-side elevator tool with the handle in an insertion alignment, FIG. 8D shows a top view of the far-side elevator tool and FIG. 8E shows a side view of the far-side elevator tool; and

FIGS. 9A-9C show different views of the far-side elevator tool consistent with FIG. 8A but in a deployed alignment where FIG. 9A shows the far-side elevator tool in an deployed alignment, FIG. 9B shows a close up view of the distal end of the far-side elevator tool with the distal paddle in an deployed alignment and FIG. 9C shows a close up view of the proximal end of the far-side elevator tool with the handle in an deployed alignment.

### DETAILED DESCRIPTION OF THE INVENTION

COPYRIGHT NOTICE: A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. The following notice applies to any software and data as described below and in the drawings hereto: Copyright © 2020-2024, Foundation Surgical Group, Inc., All Rights Reserved.

Orthopedic surgical instruments and methods of use will now be described in detail with reference to the accompanying drawings. Notwithstanding the specific examples set forth below, all such variations and modifications that would be envisioned by one of ordinary skill in the art are intended to fall within the scope of this disclosure. The surgical instruments and methods may be used with orthopedic implant systems such as, but not limited to, an intravertebral implant system for use in intravertebral applications, an intervertebral implant system for use in intervertebral applications and an implant system for arthrodesis procedures for other joints throughout the body. The surgical instruments may be used with implant systems that may comprise an orthopedic implant device such as, but not limited to, an intravertebral implant device, an intervertebral implant device or an implant device for arthrodesis procedures for other joints throughout the body.

In this description, the terms instrument(s) and tool(s) are used interchangeably and are to be interpreted as having the same meaning.

Foraminal narrowing is a specific type of spinal stenosis, a spinal condition that occurs when the open spaces between the vertebra (the foramina) narrow. The foramina are bony passageways located between the vertebrae on either side of the spine. Their primary purpose is to provide an exit path for nerves leaving the spinal cord and traveling to other parts of the body.

Minimally invasive spine (MIS) surgery focused on decompression without fusion is generally intended to relieve pressure being applied to the spinal nerves-often a result of conditions such as spinal instability, bone spurs, herniated discs, scoliosis or spinal tumors. In cases where extensive decompressions are required to accomplish the goal of relieving pain, a fusion may become necessary.

Fusion of opposing bones of a joint results in a permanent connection of the bones of the joint to eliminate motion between them. All fusions, including spinal fusion, involve techniques designed to mimic the normal healing process of broken bones where an implant device may be used to hold the vertebrae together, so they can heal into one solid and immobile unit.

For spinal applications, the disclosed surgical instruments, implant systems and methods may be used to bridge the gap between a minimally invasive decompression without fusion and more extensive decompressions requiring a fusion procedure and lead to an improved quality of life when compared to current standard surgical techniques and technology. The patient will have relief from back and/or leg pain without a loss of spine mobility, which can significantly reduce or eliminate the risk of adjacent level accelerated degeneration in the other levels of the spine. The custom alignment created with the implant device can prevent the clinical outcomes of spinal misalignment.

### Surgical Instruments and Implant System Overview:

Generally, the disclosed surgical instruments support the methods of using orthopedic implant systems. In particular, they provide the ability for the user to prepare a contralateral side of a surgical site from a position oriented on the ipsilateral side of the surgical site and provide the ability to position and implant an implant device from that position. For example, the surgical instruments are particularly helpful in surgical procedures and with implant systems that access the bone from one position or side but require site preparation on the other side of the bone. This situation is encountered with the implant devices and methods disclosed herein and in U.S. Pat. No. 11,259,936 issued on Mar. 1, 2022, pending U.S. patent application Ser. No. 18/424,843 filed on Jan. 28, 2024 and published as US2024/0164909 on May 23, 2024 and U.S. patent application Ser. No. 18/736,212 filed on Jun. 6, 2024; the entire contents of all are herein incorporated by reference. With those devices, and in those methods, the other side of the vertebral body is ideally prepared by elevating tissue and/or ligaments from the vertebral body so that a staple from the implant system can be securely anchored in the contralateral sidewall of the vertebral body. With those devices, and in those methods, the implant device is positioned and implanted from an orientation on the ipsilateral side of the bone and the implant system can be manipulated to secure the implant device to the contralateral and ipsilateral sides of the bone.

In some embodiments, the implant device generally acts as an opening wedge osteotomy spacer and uses the shape of implant components, such as cage surface planes, to alter the alignment of the vertebral body of a mammalian body.

Embodiments of the surgical instruments and implant systems for use with vertebral applications may be configured to be applied with either an anterior-to-psoas (ATP) or a direct lateral (trans-psoas) approach to the lumbar spine from the concave side of the vertebrae. The ATP approach benefits from the advantages of both anterior and lateral approaches with similar complication rates.

Embodiments of the surgical instruments and implant systems may also be used in a direct lateral approach where the far/distal/contralateral side of the vertebral body or bodies, in relation to a point of origin being the approaching side of the procedure, need more correction/separation than the near/proximal/ipsilateral side.

Embodiments of the surgical instruments and implant systems may also be used in an Anterior Lumbar Interbody Fusion (ALIF) procedure. With the ALIF procedure, surgical access to the vertebral body in the lower spine is from the front of the body (from an anterior approach) by making an incision in the belly and traversing the abdominal cavity. Additionally, it is possible to make an anterior incision and operate in the retroperitoneal space.

Embodiments of the surgical instruments and implant systems may also be used in standard posterior approaches to the spine and vertebral body. This may also include the same approach as used in a Transforaminal Lumbar Interbody Fusion (TLIF) procedure. With the posterior or posterior lateral approach, such as with the TLIF procedure, or other posterior lateral approach procedures, surgical access to the vertebral body in the lower spine is from the back of the body.

Although embodiments of the surgical instruments and implant device may be used and positioned from different planes relative to the vertebral body, some embodiments are specifically configured to be inserted and secured from a lateral or an oblique approach angle. These approach angles are particularly beneficial because they are well known to those skilled in the art and reduce the risk of complications from more traditional anterior approach procedures. Insertion from lateral and oblique angles makes it easier to avoid blood vessels, the peritoneal cavity, and abdominal muscles during the insertion procedure. This lowers the risk of injury to these vital structures (vessels, nerves & organs) and also minimizes or reduces the need for other surgical specialists, such as vascular surgeons or general surgeons, which may otherwise be required to assist in the procedure.

The ATP approach, as one particular oblique approach, may be used to access the vertebral body and implant the device. With this ATP approach, surgical access is provided to the vertebral body which can sometimes alleviate the need for an additional vascular or general surgeon. With this approach, an oblique incision is made on the patient and abdominal muscles and the retroperitoneal space are bluntly dissected to expose the psoas muscle. The psoas muscle or psoas tendon is retracted posteriorly only as required during certain portions of the procedure to define the surgical corridor and expose the spine and vertebral body for the surgery. Use to the ATP approach provides the opportunity to minimize psoas retraction.

For most of the above procedures, MIS techniques or open surgical techniques may be used which provide the benefit of minimizing patient trauma from the surgery. Special methods, tools, and implant devices, such as those disclosed herein, are helpful to accommodate the limited access and the minimal trauma desired from these techniques.

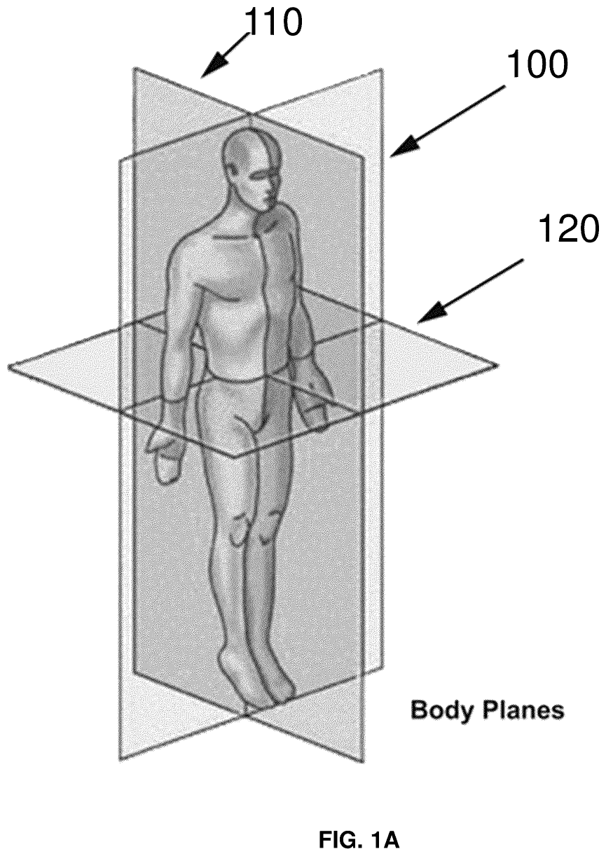

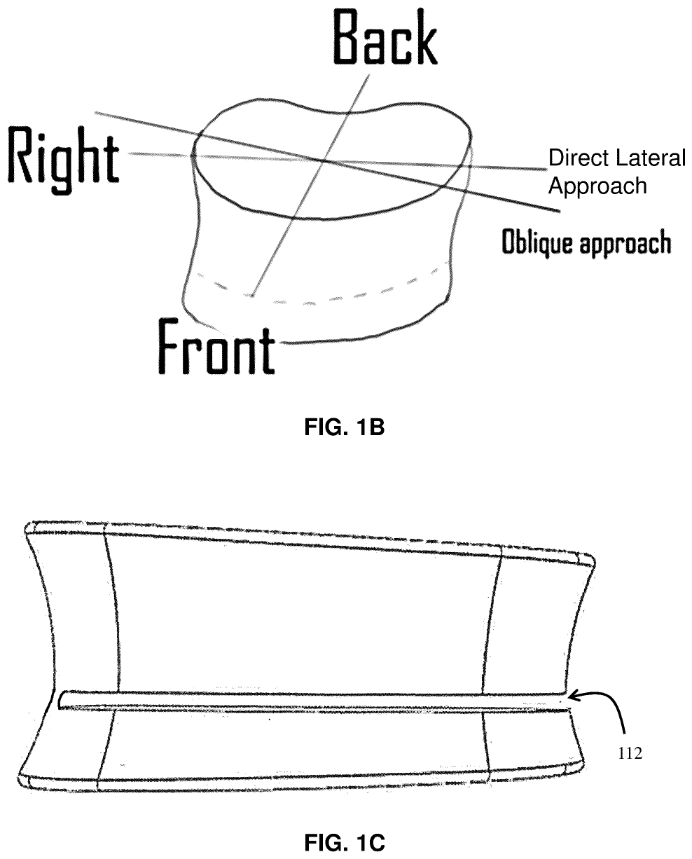

Referring to FIG. 1A showing the sagittal 110, coronal 100 and transverse 120 planes of the human body, embodiments of the surgical instruments may be used with implant systems and methods to correct alignment of the spine in the sagittal (110) and coronal (100) planes.

Referring to FIG. 1B, the surgical instruments may be used with implant systems inserted and positioned at and from different angles and orientations relative to the vertebral body. For example, some embodiments of the implant systems may be implanted through anterior (front), oblique or lateral (left/right) oriented approaches.

Examples of the surgical instruments used with implant systems configured for vertebral intrabody applications are generally used in conjunction with an osteotomy made through the vertebral body inferior to the pedicle as shown in FIG. 1C. As shown, the osteotomy 112 is cut inferior to the inferior aspect of the pedicle and level with the vertebral body's inferior endplate.

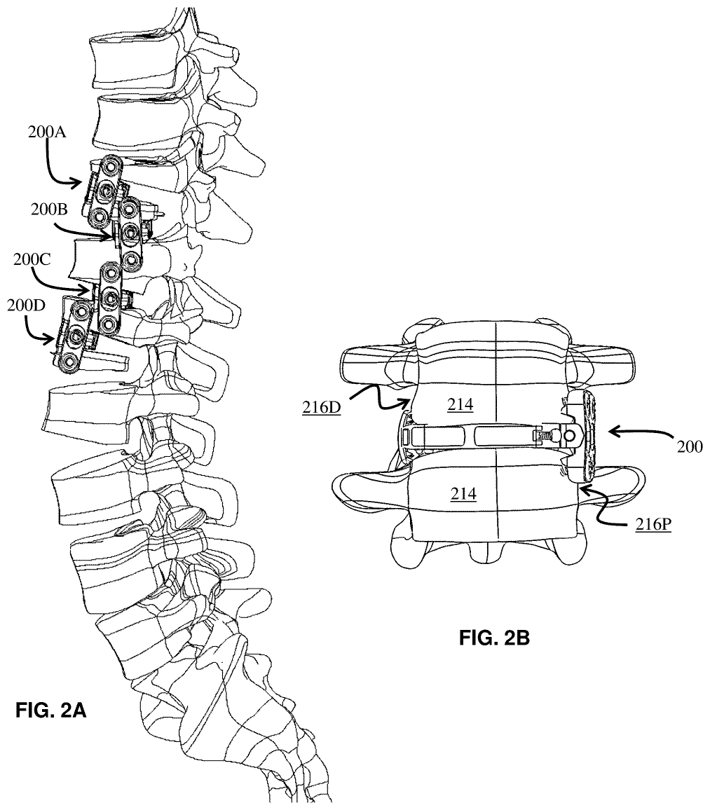

In one embodiment, the disclosed surgical instruments may be used with an implant system configured to correct vertebral body deformity in the coronal, sagittal, and axial plane (if needed). For example, FIGS. 2A and 2B show example implant devices implanted as intervertebral implants. FIG. 2B shows an anterior view of an example implant device secured between two vertebral bodies 214 and the implant is secured to the vertebral body's distal lateral side sidewall 216D with the staple and secured to the vertebral body proximal sidewall 216P with the anchor frame and anchoring elements 290. FIG. 2A shows sagittal view of the corrected spine with implant devices 200A and 200D used as an intravertebral implant devices and implant devices 200B and 200C used as intervertebral implant devices. In this example, no longitudinal rod or tether or cord is needed. It is understood that as for scoliosis corrections, the disclosed implant systems and techniques may be used with other sections of the spine such as for thoracic correction and may be used with implant systems only used as either intervertebral or intravertebral implant devices as well as in combinations.

In some embodiments, the implant system generally comprises a cage with a staple and an anchor frame. The staple and the anchor frame may be on opposing sides of the cage to secure the cage to bone. In some embodiments the staple may have features that allow the staple to be inserted, extended, deployed, and stabilized or secured to the bone. In some embodiments the anchor frame is pivotally coupled to the cage.

In some embodiments, the staple of the implant system may have extension, deployment and retracting features that allow the staple to be moved through multiple positions to secure the implant device to the bone. The movement features may allow the distal staple to be easily moved between an insertion position, an extended position, a deployed position, and a stabilized position. These different positions of the staple describe both the rotational alignment of the staple head and the location of the staple head relative to other elements of the implant device.
Insertion position: In the insertion position, alignment of the length of the staple head is in a neutral alignment, generally the orientation of the length of the staple head being coplanar or parallel to a plane extending along the transverse axis of the cage. In this position, the longitudinal location of the staple head relative to the longitudinal axis of the cage is the location as the implant device is being positioned for implanting.
Extended position: In the extended position, the alignment of the staple head is in the neutral alignment, generally parallel to the transverse axis of the cage. In this position, the longitudinal location of the staple head relative to the cage longitudinal axis is in an extended location extended away from the cage. The extended position generally extends the location of the distal staple head from the cage longitudinally so that the staple head and the staple tines extend beyond the sidewalls of the bone.
Deployed position: In the deployed position, the staple head is rotated to a non-neutral alignment that is other than parallel to the transverse axis of the cage. In this position, the non-neutral alignment of the staple head may be at any angle relative to the insertion and extended position sufficient to allow the staple tines of the staple to be positioned to engage the sidewalls of the bone. The longitudinal location of the staple head relative to the longitudinal axis of the cage is in an extended location extended away from the cage sufficient to allow the staple tines of the staple head to extend beyond the bone.
Stabilized position: In the stabilized position, alignment of the length of the staple head is not parallel to the transverse axis of the cage and sufficient to allow the staple tines of the staple to engage the walls of the bone. In this position, the alignment of the staple head is generally in the non-neutral alignment in the deployed position. In this position, the longitudinal location of the staple head relative to the cage is in a retracted location retracted towards the cage sufficient to allow the staple tines of the staple head to engage or embed themselves in the bone to mechanically engage and stabilize the staple head and the cage to the bone.

To support the above positions, the staple may comprise the staple head and a staple shaft. The staple shaft may be configured to move and rotate the staple head through the above positions.

To support the movement of the staple head through the different longitudinal locations of the above positions, the staple shaft may also be configured to move the staple head from an insertion to an extended longitudinal location by having a threaded staple shaft mate with a threaded coupling element such as a nut and rotating the coupling element to extend the staple head away from the cage. The staple shaft may also be configured to move the staple head from the deployed to a stabilization longitudinal location by having a threaded shaft mate with a threaded coupling element and rotating the coupling element to retract the staple head towards the cage.

To support the movement of the staple head through the different alignments of the above positions, the staple shaft may also have an engagement portion configured to be engaged by an instrument or engagement tool to rotate the staple shaft and staple head from the extended alignment to the deployed alignment. To maintain the alignment of the staple shaft in relation to the cage, the engagement portion may be configured to be rotationally stabilized while other implant device elements are moved.

To support the insertion of the implant system, and in particular the insertion and movement of the staple on the distal side of the bone, the far-side elevator tool is configured to clear obstructions on the distal side of the bone. This clearing of the obstructions allows the staple to move through its positions during insertion, positioning and securing of the implant device. This clearing of the obstructions also allows the staple to more securely engage the far sidewall of the bone to more securely anchor the implant device to the bone.

To support insertion of the implant system, and the movement of the staple, the insertion handle assembly is configured to couple with the implant system so that the implant system can be inserted, positioned, manipulated, and secured at the implantation site. The insertion handle assembly may also be configured to receive other instruments, such as tools to engage the staple shaft and coupling elements to position the staple and anchor the implant device to the bone.

In some embodiments, the staple is positioned on the distal side of the implant to be secured to the contralateral sidewall of the vertebral body while control of the positioning of the staple is done by manipulating system elements and features accessible on the proximal side of the implant. These features are particularly beneficial for vertebral procedures where the implants are inserted and secured from a lateral or an oblique approach angle and the implant is implanted across the vertebral body and secured to both lateral sidewalls of the vertebrae. These procedures include the ATP approach to access the vertebral body and implant the implant device.

### The Implant System:

It is understood that the disclosed implant systems, instruments and methods of use may be used with different orthopedic procedures and for different bones. For example, it is understood that the disclosed implant systems and instruments may be used as intervertebral implant systems, intravertebral implant systems and implant systems configured for use in other orthopedic procedures such as arthrodesis procedures.

For illustration purposes only, and not for limitation, an example of the implant system used for intravertebral applications will be described and referred to as a vertebral implant system, an intravertebral implant system, a vertebral implant device and an intravertebral implant device. In this illustrative example, the implant system comprises a vertebral implant device configured for use as an intravertebral implant device. For illustration purposes only, and not for limitation, examples of the implant systems as used in an intervertebral application will be described and referred to as a vertebral implant system, an intervertebral implant system, a vertebral implant device and an intervertebral implant device.

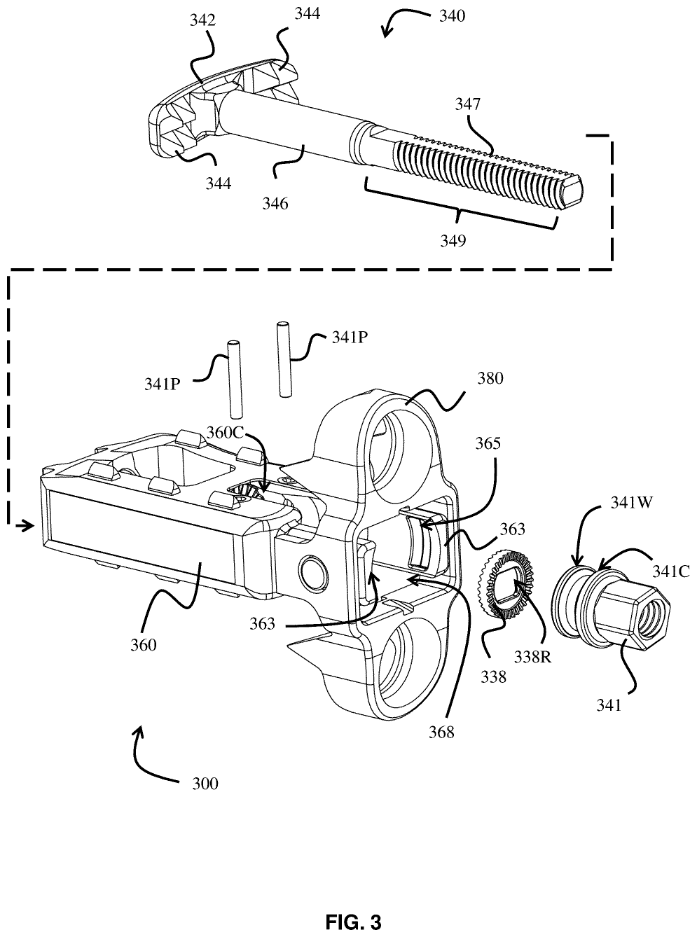

FIG. 3 illustrates an example of an implant system. Implant systems consistent with this example may be used as intervertebral implant systems, intravertebral implant systems and implant systems configured for use in arthrodesis procedures. For illustration purposes only and not for limitation, this example will be described as used as a vertebral implant system.

The vertebral implant system generally comprises a vertebral implant device comprising a cage and one or more securing element configured to secure the cage to the vertebral body. The securing element may be any suitable element or combination of elements to secure the cage and the implant device to a superior and inferior portion of the vertebral body. The securing element may comprise one or more staple received in, and rotationally and longitudinally adjustable relative to the cage.

FIG. 3 shows an example of a vertebral implant system comprising a vertebral implant device showing a perspective view of the vertebral implant device 300. FIG. 3 shows the staple in an insertion position 3421 with the staple in the insertion location and in an insertion alignment. The vertebral implant device generally comprises a staple 340, a cage 360, an anchor frame 380 and a coupling element. The cage 360 generally functions as a spacer between bones or between portions of a bone. The staple 340 generally extends through a longitudinal bore of the cage to mechanically engage a sidewall of the bone to secure it and the cage 360 to the bone. The anchor frame 380 also provides features to receive the staple 340 and secure itself and the cage 360 to the bone. Together with the staple 340, the anchor frame 380 provides a compressive force to secure the cage 360 to the bone. The coupling element, in this example a nut 341, provides adjusting device features for the implant system to extend and retract the staple shaft 346 and staple head 342 relative to the cage 360. This extension and retraction caused by the coupling element adjusts the locational relationship between the staple head 342 and the 360 and other implant device elements. Some embodiments further comprise a stop and a key to control and secure the angular alignment of the staple 340 relative to the cage 360.

The staple 340 generally comprises a staple head 342 having staple tines 344, a staple shaft 346, a coupling portion 349 and an engagement portion 347. The coupling portion 349 provides a means to extend and retract the staple head and the engagement portion 347 provides a means to rotate the staple shaft 346 and the staple head 342. The engagement portion 347 may be any configuration that provides the ability to move the staple shaft 346. In the example shown, the engagement portion 347 is a proximal portion of the staple shaft 346 having flats on its outer profile whereby the engagement portion 347 can mate with an instrument or engagement tool, such as a drive rod with a recess having mating flats, whereby the staple shaft 346 can be engaged, pushed and rotated with the tool. The engagement portion 347 may also provide a means to mate with a key 338 to engage a cage stop to influence the movement of the staple shaft 346 and the staple head 342 relative to the cage 360. The key 338 may be configured with any surface features such as for example, teeth to mechanically engage a similarly configured mating surface the cage 360. The key 338 is also configured to engage the staple shaft 346 whereby the key influences the movement of the staple shaft 346 relative to the cage 360. The key 338 may be any element configured to influence the movement of the staple shaft 346 relative to the cage 360. In the example shown, the key 338 is a radially grooved washer configured to mesh with a mating radially grooved surface on the cage 360. In the example shown, the radially grooved washer has a recess 338R with a profile including flats that mate with the flats of the engagement portion 347 of the staple shaft 346. With this example, when the engagement tool is turned, the staple shaft 346 is turned, the radially grooved washer is turned and the interaction of the radially grooved surface on the cage meshes with the radially grooved surface on the radially grooved washer to urge the engagement tool and the staple shaft 346 to lock at a finite number of predetermined rotational angles, or radial positions, relative to the cage 360.

The cage 360 generally comprises a body generally having a longitudinal axis extending from the proximal coupling with the anchor frame to the distal bore for the staple shaft and a transverse axis running generally perpendicular to the bore for the staple. The upper surface of the cage 360 defines the cage upper surface plane for the implant device and the lower surface defines the cage lower surface plane. The upper and lower surface planes, the space between them and the angles between them define the correction that can be provided through the implanting of the cage 360. The cage 360 has a through bore extending along the longitudinal axis of the cage from a first lateral side of the cage to a second lateral side. The through bore is configured to receive and retain the staple shaft 346. The central bore of the cage 360 also defines a cage retaining channel 360C to accept pins to retain the nut. The retaining channel is configured to receive and retain pins located to capture the nut 341 so that it may be rotated relative to the cage 360 and the staple shaft 346 yet maintain its approximate position relative to the longitudinal axis of the cage 360. In the example shown, the nut 341 is retained in the cage retaining channel 360C with pins 341P that are received in pin holes that extend from one surface of the cage 360 into the cage retaining channel 360C and into the nut retaining channel 341C of the nut 341. This configuration retains the nut 341 but provides for rotational freedom of movement and some minimal longitudinal translation along the longitudinal axis of the cage. The cage 360 may further comprise a cage stop (not shown) configured to mate with the engagement surface of the key. In the example shown, the cage stop comprises a radially grooved surface on the inside of the cage retaining channel 360C that engages with the radially grooved surface of the key 338 to urge the engagement tool and the staple shaft 346 to lock at predetermined rotational angles relative to the cage 360. The cage 360 may further comprises one or more staple tine recesses to receive the staple tines when the staple head 342 is in an insertion position. The cage 360 may further comprise one or more retention elements to help the upper and lower surfaces of the cage engage the bone. In the example shown, the retention element comprises one or more cleats. The cage 360 may further comprise a pivot coupler to pivotally couple the cage 360 to the anchor frame 380. The pivot coupler may be any type of coupling that allows the anchor frame 380 to move in relation to the cage 360. In the example shown, the pivot coupler comprises a cylindrical protrusion received in a through hole on the proximal end of the cage 360 whereby the anchor frame 380 pivots about an axis about ninety degrees to a longitudinal axis of the cage. In some embodiments, the anchor frame 380 pivots about an axis having a range of about 45 degrees to 90 degrees to the longitudinal axis of the cage 360.

The anchor frame 380 is operably coupled to and generally extends perpendicular to the longitudinal axis of the cage 360 and comprises securing elements to secure the cage to bone. The anchor frame 380 may have a through bore providing access for tools to access the nut 341, the proximal end of the staple shaft 346, prongs 363 and the proximal end of the cage 360. The anchor frame 380 may also have through holes to receive anchoring elements to anchor the anchor frame 380 and the cage 360 to the bone. The anchor frame may further comprise a pivot coupler to pivotally couple the anchor frame 380 to the cage 360. The pivot coupler may comprise an anchor frame pivot element and a cage pivot element, both configured to pivotally couple the anchor frame to the cage. In the example shown, the anchor frame pivot element comprises at least one cylindrical protrusion extending from the surface of the anchor frame and received in at least one cage pivot element which comprises at least one through hole on the proximal end of the cage 360 whereby the anchor frame 380 pivots about an axis about ninety degrees to a longitudinal axis of the cage.

The coupling element is generally configured to mate with the cage 360 and the staple shaft 346 to longitudinally move the staple shaft 346 and the staple head 342 through different longitudinal locations relative to the cage 360. In this example, the coupling element is a nut 341 configured to be engaged by engagement tools such as a drive rod of a staple drive handle assembly and is configured to allow access for engagement tools to engage the engagement portion 347 of the staple shaft 346. In the example shown in FIG. 3, the nut 341 is longitudinally fixed relative to the cage 360 and anchor frame 380 and engages the coupling portion 349 of the staple shaft 346 to extend and retract the staple shaft 346 and staple head 342 relative to the cage 360. The nut 341 is internally threaded to engage the threaded coupling portion 349 of the staple shaft 346 and allow the threaded portion 349 to extend through the proximal end of the nut 341 exposing the flats of the engagement portion 347 of the staple shaft 346. The nut 341 has an outer profile to be engaged by an engagement tool to rotate the nut 341. The nut 341 is received in the cage retaining channel 360C and accessible through the through bore 368 of the anchor frame 380. The nut 341 is retained in the cage retaining channel 360C by the pins 341P, a nut flange 341W and a nut retaining channel 341C. The nut flange 341W can be sized in relation to the key 338, in this example the washer, to only be inserted to a certain position in the bore of the cage. The pins 341P extend through the end bore and into the nut retaining channel 341C of the nut 341. Together, the size of the cage retaining channel 360C, the size of the nut flange 341W and the pins, hold the nut 341 within the cage retaining channel 360C of the cage 360 and allow the nut 341 to receive the threaded portion 349 of the staple shaft 346 and rotate to longitudinally move the staple shaft 346 and staple head 342 relative to the cage 360.

To help position and manipulate the implant device, the cage 360 may have one or more coupling element to couple the proximal end of the cage 360 to another tool such as an insertion handle assembly. For example, the coupling element may comprise prongs 363 configured to couple the proximal end of the cage 360 to a handle. The prongs 363 may have elements such as recesses 365 to receive handle elements and secure the handle to the prong 363. The prongs 363 and recesses 365 may be rigid so that pliant or resilient handle elements can be received in the recesses 365 to secure the handle to the prong 363 and cage 360. The prongs 363 and recesses 365 may also be pliant or resilient so that rigid handle elements can be received in the recesses 365 to secure the handle to the prong 363 and cage 360.

With the staple shaft positioned in the cage bore, the staple is rotatably coupled through a channel in the cage so that the staple can be rotated by a rotation of the shaft into the deployed position. The distal portion of the shaft is positioned through the bore of the cage that defines an opening (not shown) on the distal end of the cage. The proximal portion of the distal staple shaft is also positioned through the bore of the cage and the anchor frame so that it can be exposed to be coupled with a mating engagement tool to rotate the staple shaft.

Consistent with FIG. 3, during insertion of the intravertebral implant device, the staple head 342 is configured in an undeployed or horizontal position with staple tines 344 positioned generally parallel with the transverse surface planes of the cage 360 to allow it to fit through the osteotomy (or intervertebral space) during insertion. With the staple head 342 and the cage 360 in the implantation site, if needed, the staple head 342 may be extended to an extended position by rotating the coupling element, here the nut 341, in one direction with a mating engagement tool such as a staple drive handle assembly with a drive rod. Once the staple head 342 and cage 360 are in the correct position, the engagement portion 347 of the staple shaft 346, such as a hex or D shaped flat or other non-round profile on the proximal end of the staple shaft 346, is rotated to a deployed position by a mating engagement tool such as a staple drive handle assembly. Consistent with FIG. 3, by rotating the staple shaft 346 with the engagement portion 347 by the engagement tool, the staple tines 344 of the staple head 342 are rotated into a deployed position to engage and secure the staple tines 344 and the implant device in the vertebral body. The staple head 342 may then be retracted by rotating the nut 341 in the other direction with the engagement tool to put the staple head 342 in the anchored or stabilized position. When anchored in a stabilized position, the staple tines 344 may engage the spongy cancellous bone of the vertebral body, or they may engage the compact bone on the contralateral wall of the vertebral body.

Although the examples shown are asymmetrical or non-symmetrical about a horizontal mid-line plane of the implant device, it is understood that some embodiments of the implant components may be configured to create a symmetrical implant device about its mid-line horizontal plane.

### The Implant System Used in Intervertebral Applications:

It is understood that the above-described implant systems and methods may also be used for intervertebral applications such as an arthrodesis procedure. For example, the implant systems may be able to use the cage to separate two vertebral bodies and the distal staple and the proximal tab or other similar structure may be used to secure the implant device to the superior and inferior vertebral bodies.

Generally, these implant systems have similar features in respect to the horizontal plane so that sufficient structure is available to engage both vertebral bodies. These implant systems may also have retracting features for the distal staple or proximal tab to further secure the implant device to the walls of the vertebral bodies.

Some implant systems may be configured specifically for intervertebral use. For illustration purposes only, and not for limitation, an example of the implant system used for intervertebral applications will be described and referred to as a vertebral implant system, an intervertebral implant system, a vertebral implant device and an intervertebral implant device.

### The Implant System Used in Arthrodesis Applications:

As the above-described systems and devices may be configured for use in intervertebral or intravertebral applications, the implant systems may be used to fuse opposing bones in other body joints in applications such as an arthrodesis procedure.

For example, referring to FIG. 3, the implant system may be sized and configured so that the distal staple and proximal tines secure the implant device cage between two bones of a finger or foot/ankle joint.

### The Surgical Instruments:

Generally, the disclosed surgical instruments support the disclosed methods of using orthopedic implant systems. For example, an insertion handle assembly is disclosed to enable the implant device to be positioned and manipulated from a position oriented on the ipsilateral side of the implantation site. Additionally, a far-side elevator tool is disclosed to prepare the other side of the bone for an implant device by elevating tissue and/or ligaments from the contralateral side of the bone so that a staple from the implant system can be securely anchored in the contralateral sidewall of the bone.

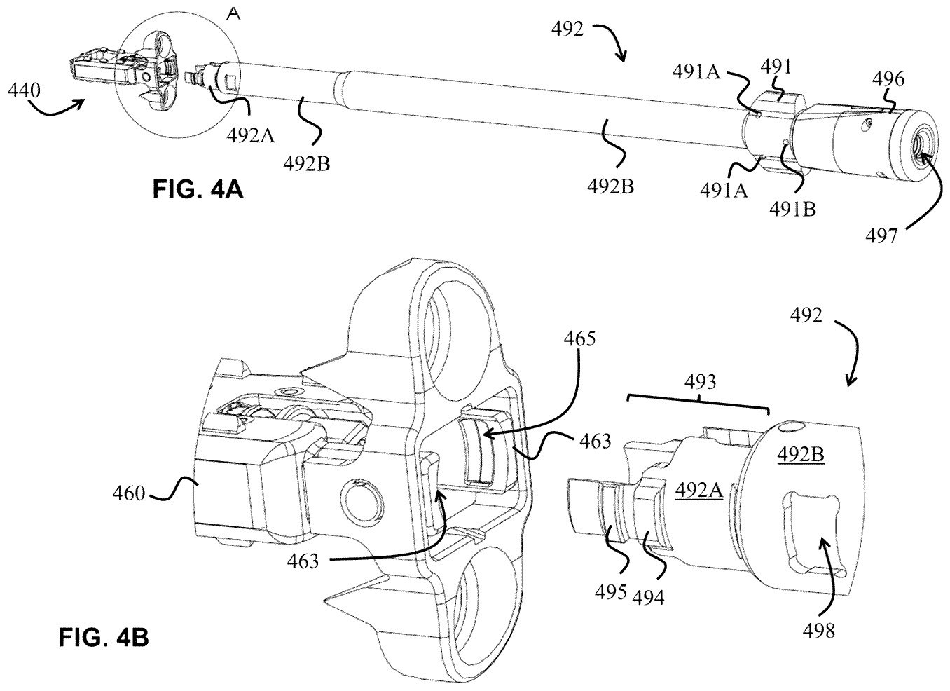

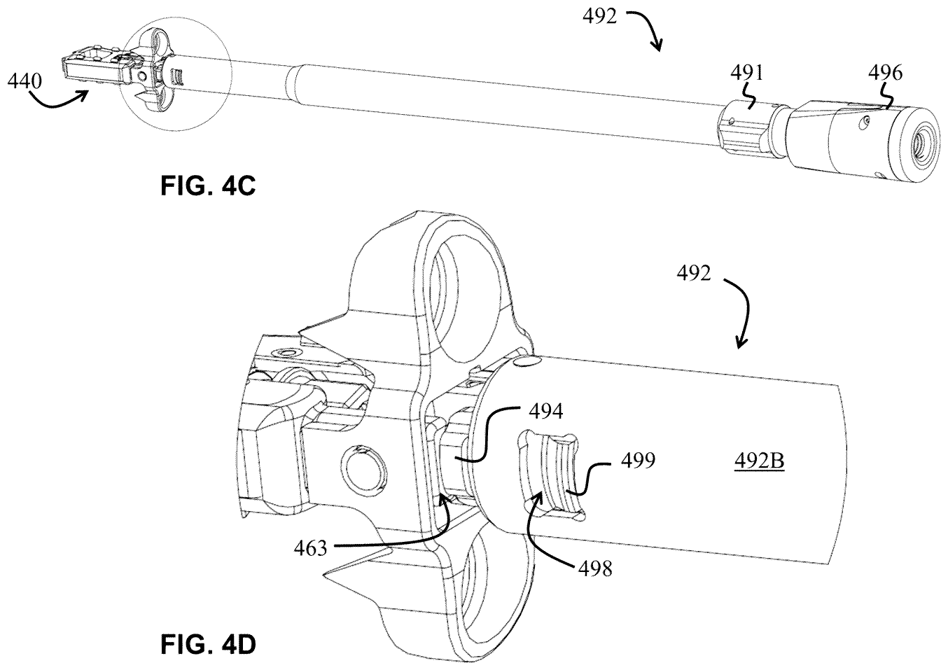

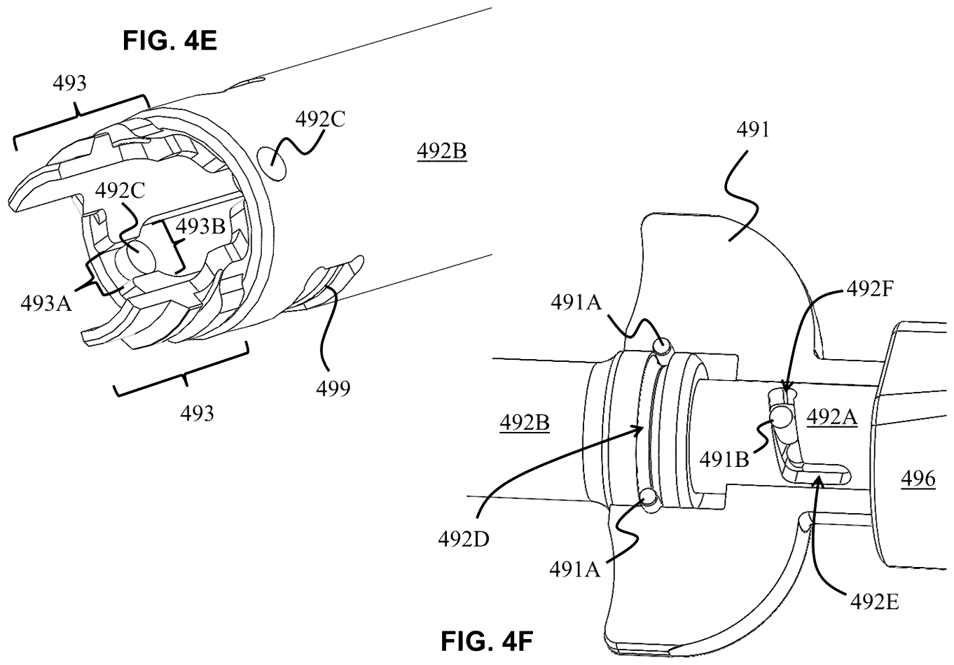

FIGS. 4A-4F show examples of instruments to be used to insert examples of the implant system. These tools may be used for the implant system whether configured for use as part of an intravertebral body implant system or as part of an intervertebral body implant system. Similar tools may be modified for use with implant systems configured to be used in arthrodesis procedures for other joints throughout the body.

FIGS. 4A-4F show different views of an insertion handle assembly 492 and implant device 440. Referring to FIGS. 4A and 4B where FIG. 4B shows detail from FIG. 4A, the distal end of the insertion handle assembly 492 is configured to engage the proximal end of the cage 460 of the implant device 440. The insertion handle assembly 492 has a switch 491 and a handle grip 496. The handle grip 496 is coupled to a cannulated inner sleeve 492A. The cannulated inner sleeve 492A is received within a handle outer sleeve 492B that is telescopically coupled to the handle grip 496. Additionally, the switch 491, the handle outer sleeve 492B, the handle grip 496, and the cannulated inner sleeve 492A are coupled such that when the switch 491 is rotated in one direction relative to the handle grip 496, the handle outer sleeve 492B is translationally locked and cannot be pulled retrograde towards handle grip 496. This is a locked position of the switch 491. When the switch 491 is rotated in the other direction, the handle outer sleeve 492B is free to translate and retract relative to cannulated inner sleeve 492A and handle grip 496. This is an unlocked position of the switch 491.

As shown in FIGS. 4A and 4B, when the switch 491 is in the position shown, the unlocked position, the cannulated inner sleeve 492A is extended further out of the handle outer sleeve 492B. This position exposes the mating prong section 493 of the cannulated inner sleeve 492A that is configured to mate with the cage prongs 463. The prong section 493 has one or more protrusion 495 to mate with recess 465 of the cage prong 463. The prong section 493 may be one or more prong resilient enough to flex between the unlocked and locked positions. In the unlocked position, resilience of the prong section 493 allows the protrusion 495 to slip into the recess 465 of the cage prong 463. The resilience also allows the prong section 493 to flex back into a locked position locking the protrusion 495 into the recess 465. The prong section 493 may also have an abutting stop 494 configured to guide the insertion of the prong section 493 into the cage prongs 463 of the cage 460. The prong section 493 may also have a lock element (see lock element 499 in FIGS. 4C and 4D) that, when the switch 491 is in the unlocked position and when in the handle outer sleeve 492B, the lock element helps urge the prong sections 493 together so that the pronged section 493 and the protrusion 495 can be flexed to unlock from the recess 465 and the cage prong 463.

FIGS. 4C and 4D show the insertion handle assembly 492 in the locked position and coupled with the implant device 440. As shown, the stop 494 has stopped and guided the insertion of the prong section into the cage prong 463. The lock element 499 may be configured to move into the handle outer sleeve 492B and a recess 498 in the handle outer sleeve 492B to help lock the relationship of the cannulated inner sleeve 492A with the handle outer sleeve 492B. As shown, the lock element 499 is in the recess 498 of the insertion handle assembly 492 allowing the prong section 493 to flex into the locked position.

FIG. 4E shows details of the distal end of the insertion handle assembly 492. The distal end of the cannulated inner rod is configured with a flex and locking mechanism to allow the prong section 493 to flex and lock relative to each other. As shown, the flex mechanism allowing a flex is a gap 493A and 493B between prongs that allows the prong sections 493 to flex towards each other. Also shown is a locking mechanism comprising one or more lock pin 492C sized to fit within gap 493A whereby when the switch is in the locked position, the lock pin 492C fits within the gap 493A preventing the prong sections 493 from flexing too far towards each other. This locking mechanism is configured to keep the prong sections 493 separated and secured in the mating elements of the implant device.

FIG. 4F shows a cut-away view of the switch 491 showing components of the insertion handle assembly 492 and its components. The switch 491 is configured with a guide mechanism to allow the switch to move the handle outer sleeve 492B relative to the cannulated inner sleeve 492A. As shown, the guide mechanism comprises pins 491A that extend through the switch 491 and mate with channel 492D in the handle outer sleeve 492B. The guide mechanism also comprises pin 491B extending into the switch 491 and into channel 492E of the cannulated inner sleeve 492A. The guide mechanism allows the switch 491 to be moved which moves the cannulated inner sleeve 492A relative to the handle outer sleeve 492B. For example, the switch 491 may be rotated in one direction with pin 491B guided by channel 492E to extend the switch 491 away from the handle grip 496 while the pins 491A extend the handle outer sleeve 492B away from the handle grip and retract the cannulated inner sleeve 492A relative to the handle outer sleeve 492B. Retracting the cannulated inner sleeve 492A retracts the distal prong sections 493 relative to the handle outer sleeve 492B urging the prong sections 493 to be locked with the lock element 499 and the lock pins 492C. Rotating the switch 491 in the other direction extends the cannulated inner sleeve 492A relative to the handle outer sleeve 492B and allows the distal prong sections 493 to flex and move in or out of the mating elements of the implant device. The insertion handle assembly 492 may also comprise a locking mechanism to retain the switch 491 in the locked position. As shown, the locking mechanism may comprise a channel portion 492F sized to retain the pin 491B and the switch 491 in the locked position. The locking mechanism may also comprise a coil spring (not shown) positioned between switch 491 and handle grip 496 such that when the switch 491 is urged into the locked position.

Operationally, for this example embodiment, the insertion handle assembly 492 may start in the locked position. To couple the implant device 440 to the insertion handle assembly 492, the switch 491 is turned 90 degrees to the unlocked position and the handle outer sleeve 492B is retracted back over the cannulated inner sleeve 492A. Retracting the handle outer sleeve 492B allows the lock pin 492C to retract from the smaller gap 493A into the larger gap 493B allowing the prong sections 493 to flex towards each other. The lock element 499 now within the inner diameter of the handle outer sleeve 492B may also help urge the prong section 493 to flex towards each other. The distal end of the insertion handle assembly 492 is then coupled to the implant device 440 by inserting the prong section 493 into the cage prongs 463 of the implant device 440 and the protrusions 495 are secured in the recesses 465. The switch 491 is then rotated into the locked position which prevents translation of the handle outer sleeve 492B back over the cannulated inner sleeve 492A, prevents flex of the prong sections 493 with the lock pin 492C, locks lock element 499 in the recess 498, and locks the protrusions 495 in the recesses 465. The implant device 440 is then positioned at the implantation site by manipulating the insertion handle assembly 492 which manipulates the implant device 440.

With the insertion handle assembly 492 coupled to the implant device 440 and the implant device 440 at the implantation site, the insertion handle assembly 492 may also be used to guide other surgical instruments and tools. For example, as shown in FIG. 4A, the insertion handle assembly 492 may be cannulated with a channel 497 that can receive tools and guide them to the implant device and implantation site. For example, the channel 497 may allow tools such as a staple drive handle assembly to extend through it and engage and manipulate elements of the implant device 440 such as the staple, the staple shaft and engagement elements of the device.

With the implant device 440 secured to the bone, the insertion handle assembly 492 may be uncoupled from the implant device 440. This can be done by rotating the switch into the unlocked position, which retracts the handle outer sleeve 492B back over the cannulated inner sleeve 492A to allow the protrusions 495 to disengage from the cage prongs 463 and allows the insertion handle assembly 492 to be removed from the implant device 440 and implantation site.

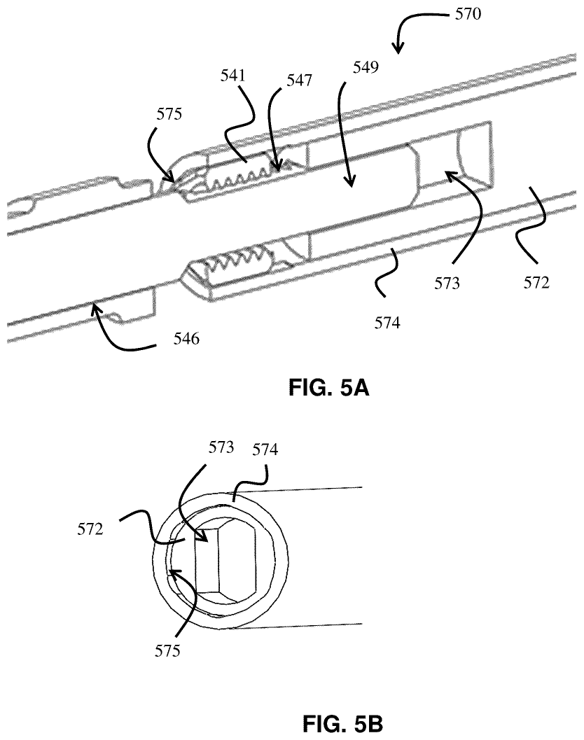

FIGS. 5A and 5B show elements of a staple drive handle assembly 570 as part of an example engagement tool. In the example shown, the staple drive handle assembly 570 has an inner drive rod 572 and an outer drive rod 574 to engage portions of the staple shaft 546. FIG. 5A shows an end view of the staple drive handle assembly 570 and how the drive rods, as engagement tools, engage the staple shaft 546. The inner drive rod 572 fits within the outer drive rod 574 and its distal end is configured to engage the engagement portion 547 of the staple shaft 546. The mated coupling of the inner drive rod 572 and the shaft engagement portion at 573 with the staple shaft engagement portion 547 allows the staple shaft 546 to be rotated so that the staple can be aligned and positioned in the deployed position. In this example, the staple shaft engagement portion 547 is a portion of the staple shaft with flats and the shaft engagement portion 573 of the inner drive rod 572 is a mating recess with flats. The outer drive rod 574 is configured to engage the coupling element. As shown, the engagement is the outer drive rod with the outer surface of the coupling element, here a threaded nut 541, that mates with the coupling portion, here a threaded portion, of the staple shaft 546. The coupling of the outer drive rod 574 and the nut 541 at the nut engagement portion 575 allow the nut 541 to be rotated by the outer drive rod 574 when the staple drive handle is rotated. This rotation and the mated threading of the staple shaft 546 and nut 541 allows the staple shaft 546 to be slidably extended, rotated and retracted in relation to the cage within the cage through bore. Rotation of the nut 541 in one direction extends the staple drive shaft 546 relative to the cage and rotating the nut in the other direction retracts the staple drive shaft 546. Rotating the staple shaft 546 also rotates the distal staple through its different positions. The inner drive rod 572 and outer drive rod 574 may be frictionally engaged to allow a turn of the staple drive handle to turn both drive rods until the staple shaft 546 positions the staple in the “deployed” position. This positioning may be assisted by the stops and keys on the staple shaft and/or the cage or other elements that can be configured to stop rotation of the staple shaft 546 when the staple is in the deployed position. Once the staple is in the deployed position, the outer drive rod 574 may be rotated by the staple drive handle to turn the nut 541 and retract the staple shaft 546 and staple. FIG. 5B shows an example of the end of the staple drive handle assembly with the inner drive rod with a shaft engagement portion 573 shaped to mate with the coupling portion 549 of the staple shaft 546 and the outer drive rod having a nut engagement portion 575 shaped to mate with the exterior surface of the nut 541.

In some embodiments, the staple drive handle assembly 570 may be configured to assist in the deployment of the staple in other ways. For example only, referring to FIG. 5A, the distal end of the staple drive handle assembly 570 may be configured to push on an element, such as the nut 541, to push the staple shaft 546 and the staple away from the cage and outside of the osteotomy so that it can be rotated and retracted to secure the cage to the vertebral body. In this embodiment, the nut is able to be moved longitudinally for some distance in the cage retaining channel. As shown, the configuration may include using the distal end of the inner drive rod 572 to push on the nut 541 which pushes the staple shaft 546 and staple further outside of the cage. The movement of the nut 541, staple and staple shaft 546 may be limited in this configuration by physical limitations. For example, as shown, the distal end of the outer drive rod 574 or the nut 541 may be physically limited to move only a certain distance by hitting a physical stop such as the proximal end of the cage or other component. In some embodiments, physical stops for the nut 541 in cooperation with the rotation and the threading of the staple shaft 546 and nut 541 may allow the staple shaft 546 to be retracted back towards the cage.

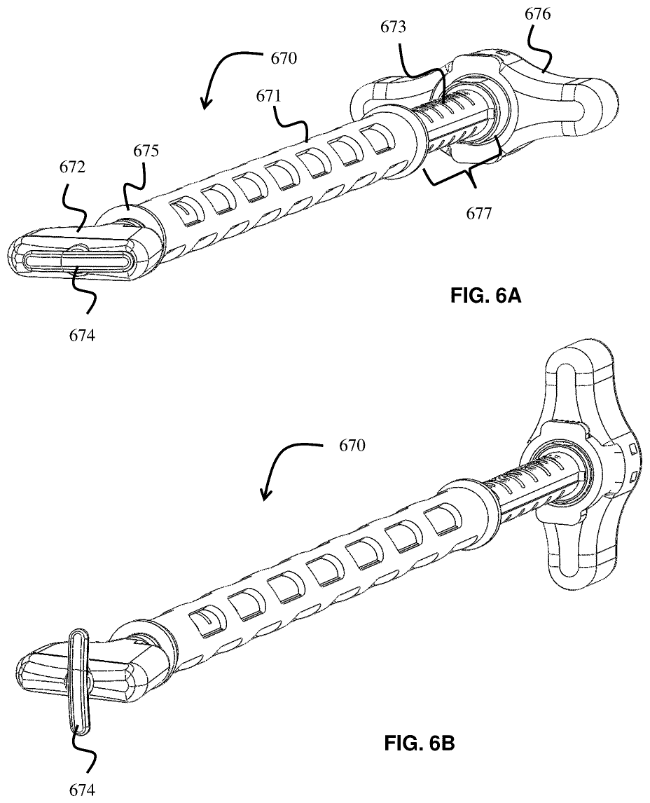

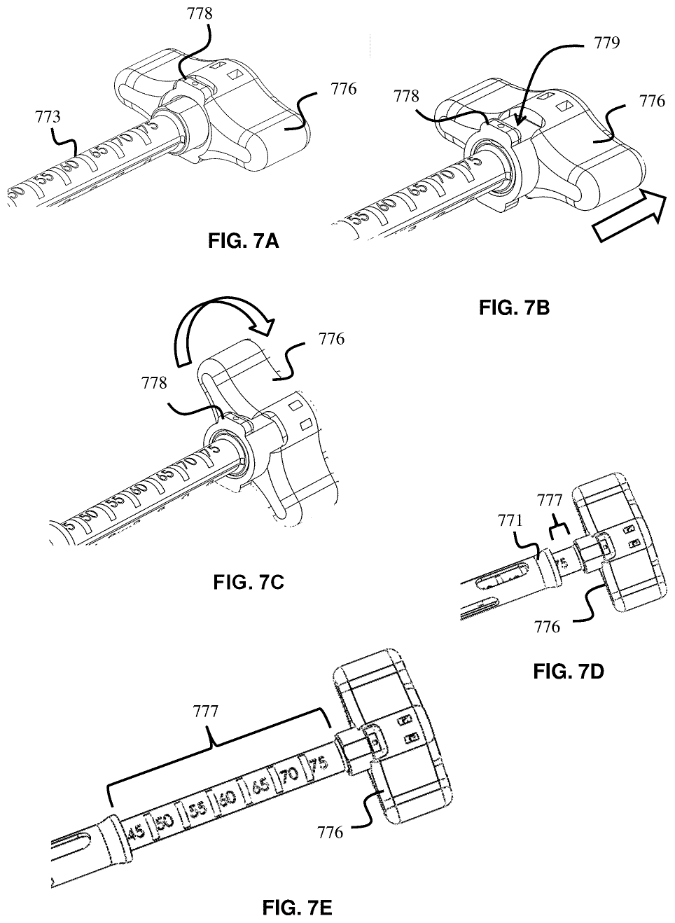

FIGS. 6A and 6B show different views of an example of a far-side elevator tool 670. The far-side elevator tool 670 generally is used to prepare the insertion site for the implant device. The far-side elevator tool 670 comprises a shell 672, a distal paddle 674, a positioning grip, an inner shaft 673 and a paddle handle 676. The positioning grip, here outer sleeve 671, is coupled to the shell 672 to control its position. A paddle shaft (not shown) is coupled to the distal paddle 674 and the paddle handle 676 to allow the paddle handle 676 to control the rotational position of the distal paddle 674 (see FIG. 6B). The distal paddle 674 may have one or more arms extending from its rotational axis. As shown, the distal paddle 674 has two arms. The distal paddle 674 and its one or more arms may move through different rotational positions such as but not limited to about 45 degrees, about 90 degrees, about 180 degrees or rotate through an unconstrained 360 degrees. In embodiments, the combination of the number of arms and the degree of rotation of the arm or arms provide rotation of the arm or arms to cover 360 degrees about the rotational axis of the distal paddle. The inner shaft 673 is received in the outer sleeve 671 and allows for the extension of the inner shaft 673 in and out of the outer sleeve 671. The inner shaft 673 is configured to have an exposed measuring portion 677 beyond the outer sleeve 671. The measuring portion 677 is marked with calibrations so that if an outer sleeve stop 675 is pushed up against a vertebral body, the calibration identifies the distance from the edge of the vertebral body to a tool component such as the distal paddle 674 (see FIGS. 7D and 7E). This distance may be used to ensure the distal paddle 674 is positioned properly when it is rotated to clear obstructions on the contralateral or far-side of the vertebral body.

Referring to FIGS. 7A-7E, the paddle handle 776 is configured to lock relative to the positioning grip, her outer sleeve 771, and the inner shaft 773 with a shaft lock 778. The shaft lock 778 may be disengaged by pulling the handle portion of the paddle handle 776 away from the inner shaft 773, opening a lock gap 779 to allow the handle portion to rotate and rotate the paddle.

FIGS. 7A-7E show different views of an example of a far-side elevator tool where FIG. 7A shows a paddle handle in an insertion alignment, FIG. 7B shows the paddle handle in an extended and unlocked position, FIG. 7C shows the paddle handle rotating and FIGS. 7D and 7E show the measuring portion 777 exposed.

Like the position of the staple, the distal paddle can be manipulated through different positions. In some embodiments, the distal paddle of the far-side elevator tool may have extension, deployment and retracting features that allow the distal paddle to be moved through multiple positions to prepare the implantation site for the implant device. The movement features allow the distal paddle to be easily moved between an insertion position, an extended position, a deployed position, and a removing position. These different positions of the distal paddle describe both the rotational alignment about a rotational axis of the distal paddle head and the location of the distal paddle head relative to other elements of the far-side elevator tool.
Insertion position: In the insertion position, alignment of the length of the distal paddle head is in a neutral alignment, generally the orientation of the length of the distal paddle head being coplanar or parallel to a plane extending along the transverse axis of the shell. In this position, the longitudinal location of the distal paddle head relative to the longitudinal axis of the shell is the location as the distal paddle is being positioned for operation. In some embodiments, the longitudinal location of the distal paddle head is generally positioned in a non-extended/insertion location close to the distal end of the shell. In some embodiments, the insertion location of the distal paddle head position may have the distal paddle head in an extended location extended away from the shell.
Extended position: In the extended position, the alignment of the distal paddle head is in the neutral alignment, generally parallel to the transverse axis of the shell. In this position, if needed, the longitudinal location of the distal paddle head relative to the shell longitudinal axis is in an extended location extended away from the shell. The extended position generally extends the location of the distal paddle head from the shell longitudinally so that the distal paddle head and the distal paddle arms extend beyond the sidewalls of the bone.
Deployed position: In the deployed position, the distal paddle head is rotated to a non-neutral alignment that is other than parallel to the transverse axis of the shell. In this position, the non-neutral alignment of the distal paddle head may be at any angle relative to the insertion and extended position sufficient to allow the distal paddle arms of the distal paddle to be positioned to elevate or remove obstructions on the sidewalls of the bone. The longitudinal location of the distal paddle head relative to the shell or the proximal side of the bone is in an extended location extended away from the shell or proximal side of the bone sufficient to allow the distal paddle arms of the distal paddle head to extend beyond the bone so that it can engage and prepare the contralateral side of the bone. Preferably, the non-neutral alignment in the deployed position allows the rotation of the paddle arms to cover about 360 degrees of the bone surface through rotation from the neutral alignment to maximize the area of preparation that can be done with the bone. For example, for a paddle head with two distal paddle arms, the non-neutral alignment in the deployed position allows the rotation of the paddle arms through about 180 degrees of rotation each.
Removing position: In the removing position, alignment of the length of the distal paddle head is parallel to the transverse axis of the shell and sufficient to allow the distal paddle arms of the staple to not engage the walls of the bone when the far-side elevator tool is removed from the implantation site. In this position, the alignment of the distal paddle head is generally in the neutral alignment in the deployed position. In this position, the longitudinal location of the distal paddle head relative to the shell is in a retracted location retracted towards the shell sufficient to allow the distal paddle arms of the distal paddle head to avoid engagement with the bone as the far-side elevator tool is removed.

To support the above positions, the distal paddle may comprise the distal paddle head and a distal paddle shaft. The distal paddle shaft may be configured to move and rotate the distal paddle head through the above positions. For example, the distal paddle shaft may be rigidly coupled to the distal paddle head and configured to move the distal paddle head from an extended position to a deployed position by a rotation of the shaft and the distal paddle head. As another example, the distal paddle shaft may be configured to move the staple head from an insertion position to an extended position by slidably moving the distal paddle shaft through a bore of the shell and extending the distal paddle head away from the shell. The distal paddle shaft may also be configured to move the distal paddle head from a deployed position to a removing position by rotating and slidably moving the distal paddle shaft through a bore of the shell and retracting the distal paddle shaft and distal paddle head towards the shell.

In some embodiments, the distal paddle is manipulated on the contralateral side of the implantation site yet control of the positioning of the distal paddle is done by manipulating system elements and features accessible on the proximal side of the implant. These features are particularly beneficial for vertebral procedures where the implants are inserted and secured from a lateral or an oblique approach angle and the implant is implanted across the vertebral body and secured to both lateral sidewalls of the vertebrae. These procedures include the ATP approach to access the vertebral body and implant the implant device.

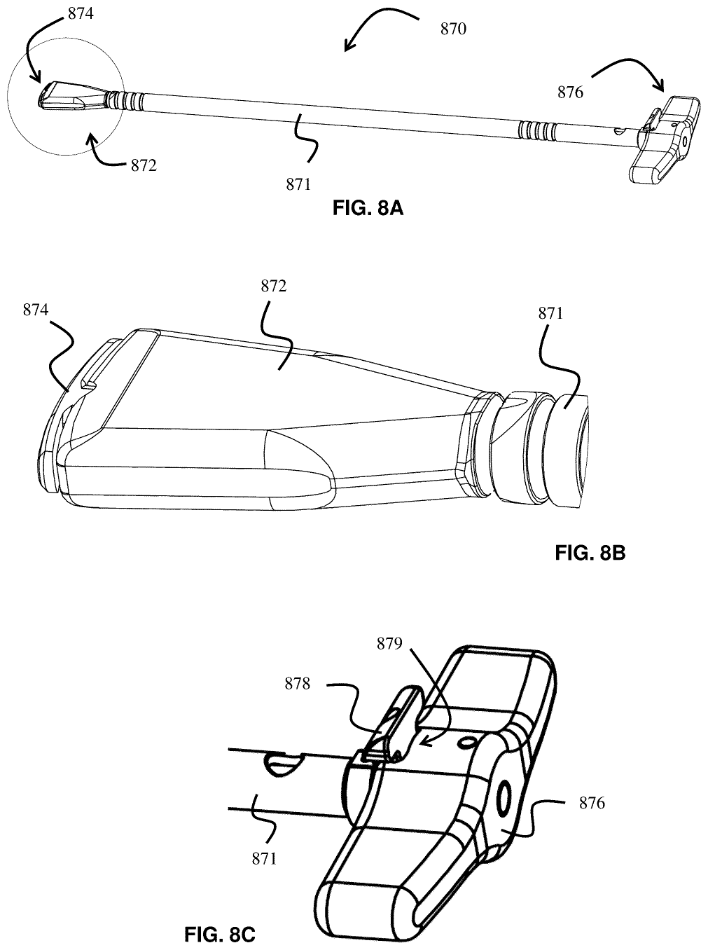

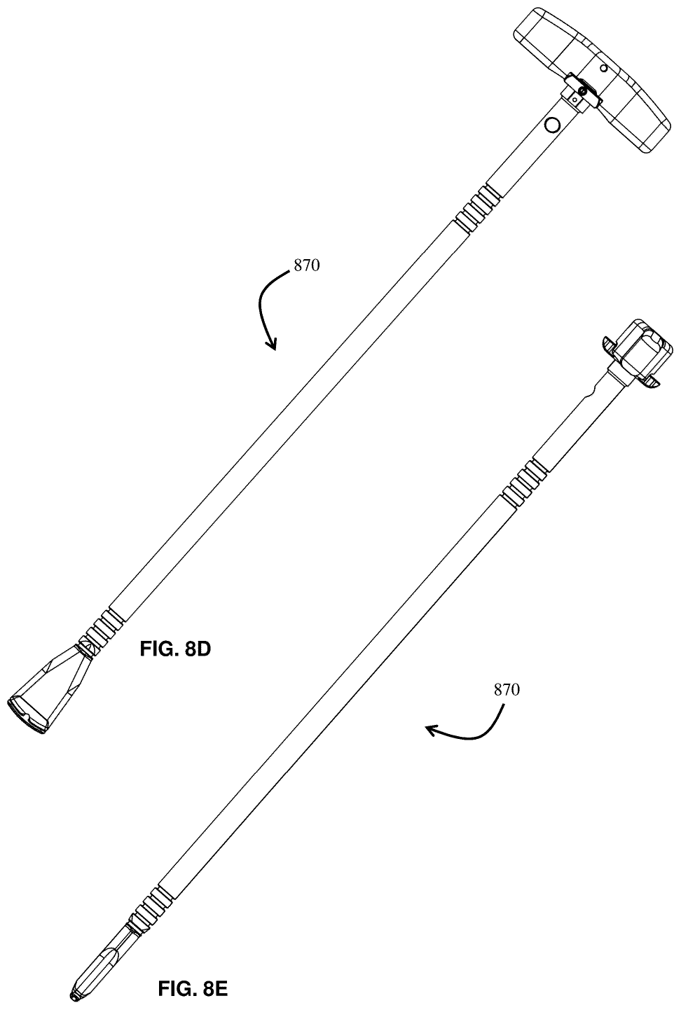

FIGS. 8A-8E show different views of another example of a far-side elevator tool. FIG. 8A shows the far-side elevator tool 870 in an insertion alignment with a paddle handle assembly 876 on a proximal end of the tool, a positioning grip 871, a shell 872 and a distal paddle 874 on the distal end of the tool. FIG. 8B shows a close-up view of the distal end of the far-side elevator tool 870 with the distal paddle 874 in an insertion alignment. As shown in FIG. 8B, in some embodiments, the shell 872 is shaped to extend across the vertebral body in either intervertebral or intravertebral implant procedures. This shape is generally thin enough to fit between vertebral body portions but wide enough and long enough to provide some stabilization of the distal end of the elevator tool during use. As shown, the distal paddle 874 is generally aligned with the upper and lower surface of the shell 872. FIG. 8C shows a close-up view of the proximal end of the far-side elevator tool 870 with the paddle handle assembly 876 in an insertion alignment that positions the distal paddle 874 in the insertion alignment. FIG. 8C also shows the shaft lock 878 and the lock gap 879. As shown, the shaft lock 878 is engaged in the lock gap 879 locking the paddle handle assembly 876 and the distal paddle 874 in a locked position. FIG. 8D shows a top view of the far-side elevator tool 870 and FIG. 8E shows a side view of the far-side elevator tool 870.

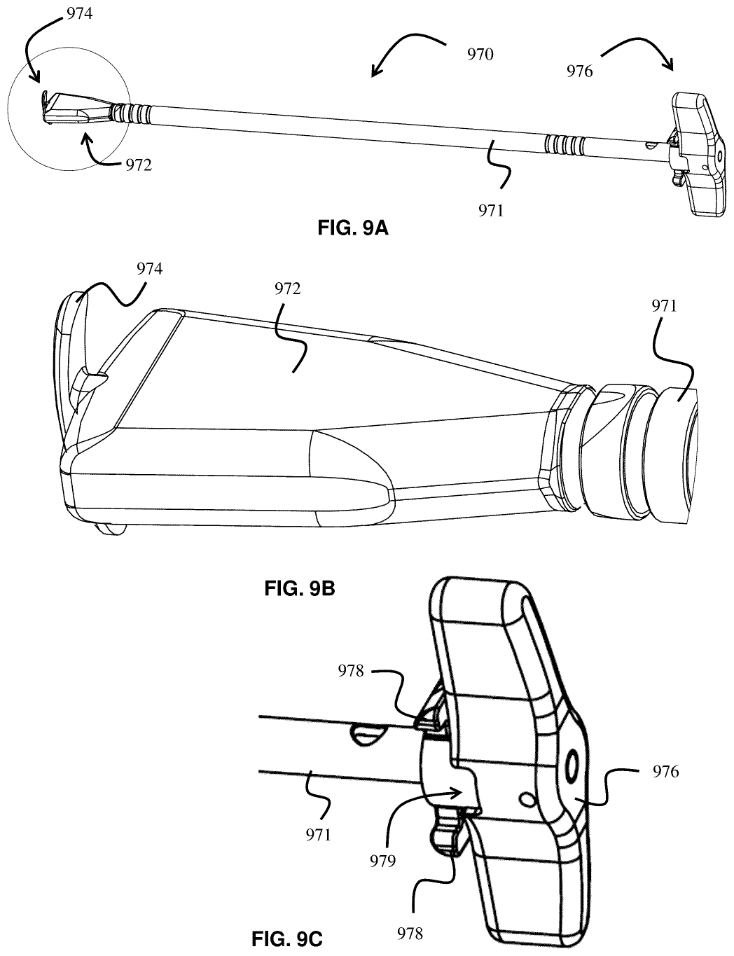

FIGS. 9A-9C show different views of the far-side elevator tool consistent with the embodiment of FIG. 8A but in a deployed alignment. FIG. 9A shows the far-side elevator tool 970 with the distal paddle 974 and the paddle handle assembly 976 in a deployed alignment. The distal paddle 974 is moved into this position by having the paddle handle assembly 976 coupled by a rod (not shown) extending through a receiving portion of the positioning grip, here outer sleeve 971, and a receiving portion of the shell 972 so that a rotation of the paddle handle assembly 976 rotates the distal paddle 974. FIG. 9B shows a close-up view of the distal end of the far-side elevator tool 970 with the distal paddle 974 in a deployed alignment with the shell 972 still generally in a horizontal position. FIG. 9C shows a close-up view of the proximal end of the far-side elevator tool 970 with the paddle handle assembly 976 in a deployed alignment. FIG. 9C also shows the shaft lock 978 and the lock gap 979. As shown, the shaft lock 978 is disengaged from the lock gap 979 in an unlocked position allowing the paddle handle assembly 976 to rotate and rotate the distal paddle 974. The shaft lock 978 may be disengaged by pulling the handle portion of the paddle handle assembly 976 away from the outer sleeve 971, opening a lock gap 979 to allow the paddle handle assembly 976 to rotate.

It is understood that the paddle handle assembly may rotate the distal paddle 974 through use of other mechanisms such as a trigger mechanism. For example, the paddle handle may be operably coupled to the paddle with a trigger mechanism and when the paddle handle is engaged, the trigger mechanism rotates the paddle to clear an obstruction or elevate a tissue on the contralateral or opposite side of the bone.

### Example Surgical Instruments and an Example Implant System in Operation:

The implant device generally uses the exterior surface planes of the cage to alter the alignment of skeletal components of a mammalian body. Referring to FIG. 1A, the disclosed implant device primarily provides adjustment of the spine in the sagittal (110) and coronal (100) planes and combinations of these two planes.

Referring to FIG. 1B, the implant device, when used as a vertebral implant, is intended to be used on the vertebra and may be inserted from different orientations. As shown, the implant device may be inserted from a posterior approach, from a lateral/side approach, from an anterior/front approach, or from an oblique approach which is understood to be a direction between posterior and lateral or between lateral and anterior. The implant system may also be applied to different portions of the spine (thoracic or lumbar).

FIGS. 2A and 2B illustrate examples of the implant device and how it may be implanted in the vertebral body.

Described below in detail is an example anterior-to-psoas (ATP) approach for a vertebral implant system used in an intravertebral procedure which is conducted oblique to the coronal plane for creating a vertebral body osteotomy and then for placing the implant within the vertebral body for correction in the coronal plane. The instruments and procedure can easily be adapted by the skilled artisan to accommodate approaches such as posterior, posterior-lateral, anterior, lateral, oblique and/or ATP. With the disclosed systems and methods, spine correction is established while the spine flexibility thru the disc and facet joints is retained, and the vertebral body then fuses in a period of time, such as 12 weeks, for a solid corrected vertebral structure.

An example method of implanting one example of the implant system consistent with the implant system of FIG. 3 generally comprises the steps described below.

A far-side retractor tool may be positioned around the implantation site to protect tissue before or during the implant procedure.

An osteotomy is made through the vertebral body from the concave side and inferior to the inferior aspect of the pedicle, preferably as parallel as possible to the inferior endplate. The osteotomy may be made from the concave side of the vertebrae. If only coronal correction is required, the far side cortex may be green-stick fractured. If coronal and sagittal correction is required, then a majority of the body may be cut, and the posterior lateral corner may be green-sticked, or a complete osteotomy may be performed. Should the skilled artisan decide to approach the spine from the convex side, this is also possible and within the artisan's skill set.

For a complete osteotomy, for other complete through cuts through the bone, or for implants between bones, a footprint sizer tool may be used to size the implant device size to be used. Additionally, for example as shown in FIGS. 6A and 6B, a far-side elevator tool may be inserted into the space between the bones and the handle may be turned to turn the paddle on its distal end to clear obstructions on the far side of the implant location.

Prior to implanting the implant device, the far-side elevator tool may be used to remove obstructions from and prepare the contralateral sidewalls for engagement with the staple of the implant device. The far-side elevator tool has the switch in the locked position with the distal paddle in an insertion position aligned between the surface planes of the shell. This insertion position allows the distal paddle and shell to be positioned in the implantation site (osteotomy or disc space) with the positioning grip. The shell is positioned in the osteotomy or disc space and the paddle handle assembly is unlocked from the shaft lock and rotated so that the distal paddle is in a deployed position and rotatable against the contralateral sidewall of the vertebral body. For the example shown, the paddle handle assembly is typically rotated at least 180 degrees to have the distal paddle clear the obstructions. If needed, the distal paddle may be moved to an extended position by moving the distal paddle and shell further into the osteotomy or disc space so that the distal paddle engages the contralateral sidewall of the vertebra. Additionally, the positioning of the distal staple may be guided by resting the outer sleeve stop against the proximal sidewall of the vertebra and the measuring portion of the inner shaft can be used to determine the location of the distal staple relative to the ipsilateral sidewall. With the contralateral sidewall obstructions cleared, the distal staple is moved into the removing position and locked. The far-side elevator tool is then removed.

Before or after use of the far-side elevator tool, the insertion handle assembly is secured to the implant device. Consistent with the examples in FIGS. 4A-4F, the insertion handle assembly is secured to the implant device by moving the switch of the insertion handle assembly into an unlocked position which allows the distal prongs to be flexed towards each other. With the distal prongs able to flex, they are inserted into the recesses of the implant device cage prongs. While in this position, the switch is rotated into a locked position which locks the distal prongs of the insertion handle assembly with the implant device.

With the insertion handle assembly secured to the implant device, the implant device is advanced and positioned in the osteotomy or disc space at the insertion site. Prior to implanting, the cage may be filled with bone graft material of choice. During this step, the staple is in an insertion position to pass through the osteotomy or disc space with the cage.

With the implant device at the implantation site, the staple drive handle assembly is positioned through the insertion handle assembly and the distal end of the staple drive handle assembly is positioned over the engagement portion of the staple shaft and nut (for example only, see example at FIG. 5A) so that it can engage and rotate the staple shaft and the nut independently. If needed, the staple shaft and staple may be moved to an extended position further away from the distal end of the cage so that the staple is positioned outside of the osteotomy and beyond the opposite side border of the contralateral vertebral walls. This extended position may be accomplished by (1) holding the staple shaft drive rod (e.g., inner rod) of the assembly so that the staple shaft does not rotate and (2) rotating the nut drive rod (e.g., outer rod) of the assembly to turn the nut and have the nut engage the threads of the staple shaft to extend the staple shaft and staple away from the cage. The use of multiple nested drive rods can allow the rotation of one drive rod while not rotating the other (for example only, see examples in FIGS. 5A, 5B). Once the staple is in the extended position, the staple drive handle assembly engaging the engagement portion of the staple shaft is rotated to rotate the staple shaft and position the staple into the deployed position. The rotational movement may be influenced by the key and the cage stop to stop rotation or increment the rotation through rotational angles. For example, the cage stop and the key may increment the rotation of the staple through degree increments such as for example only 6 degree increments. While holding the inner drive rod of the staple drive handle steady, to maintain the alignment of the staple head, the drive rod of the staple drive handle assembly engaging the nut may then be rotated in the other direction to turn the nut and retract the staple shaft and head towards the cage to have the staple tines and the tab to engage the contralateral wall of the vertebral body and secure the implant device to the vertebral body in the stabilized position. This retraction also locks the key and cage stop to maintain the alignment of the staple head. The near side of the implant device may help secure the implant device with a proximal tab or an anchor frame. The staple drive handle assembly may then be removed, or it may stay engaged to help stabilize the implant device.

For implant systems that secure the anchor frame and implant device to the vertebral body directly through holes in the anchor frame, anchoring members may be positioned and secured to the vertebral body. Once the cage and anchor frame are positioned, the anchoring members (e.g., bone screws) may be inserted through the anchor frame and through holes using a tool such as but not limited to a screwdriver. With the insertion handle assembly still coupled to the implant device, all bone screws may be inserted into the vertebral body.

The insertion handle assembly may then be removed.

For implant systems with cage screws, the cage screws may incorporate an anti-backout thread design (e.g., spiral-lock) or anti-backout elements to prevent loosening or disengagement of the cage from the anchor frame once it is implanted.

For some embodiments, a bone screw anti-backout feature may be positioned over the heads of the bone screws to prevent them from backing out.

For some embodiments, should additional bone graft material be desired within the cage, additional bone graft material may be delivered into the cage cavity through a cage recess/hole.

Appropriate instrumentation as known to a skilled artisan would be provided to the surgeon to assist and facilitate every step of the above implantation procedure. These instruments would include but not be limited to, cutting guides, cage introducer/retractor, cage inserter/holders, sizing template, drill template, drill bits, plate holder/introducer and screwdrivers. A skilled artisan would also adapt these instruments appropriately to accommodate the desired surgical approach: anterior-to-psoas (ATP), anterior, posterior, posterior-lateral, oblique, or direct lateral.

In some embodiments, the implant device may provide additional correction in the sagittal plane. In these embodiments, the cage surface planes may have different angles between them to affect correction in the coronal and sagittal plane. The transverse angle of the cage may additionally provide some correction in the sagittal plane when implanted from a lateral approach.

In some embodiments, the implant device may be inserted from other approaches or may be used to alter alignment in other planes. With other approaches, the general method of inserting and securing the implant device is similar to the methods above. The different approach direction may require different configurations of the implant device and associated instrumentation so that the exterior surface planes of the implant device provide the desired alteration in superior endplate surface plane and the inferior endplate surface plane of the vertebra in the appropriate plane.

### Example Surgical Instruments and an Example Implant System in Operation:

The above procedures generally describe use of the implant system for use as an intravertebral implant system. For implant systems used as an intervertebral implant system, similar tools and methods may be used. Rather than implanting the implant device between the two sections of one vertebral body after an osteotomy, these implant systems are implanted between the end plates of two opposing vertebral bodies.

### Example Surgical Instruments and an Example Implant System in Operation:

For implant systems configured for use with other joints in an arthrodesis procedure, similar tools and methods may be used as those described for vertebral implant systems. The tools may be sized differently to accommodate the size and location of the joint being fused.

It is also understood that these methods may be used in applications without osteotomies.

Although this invention has been described in the above forms with a certain degree of particularity, it is understood that the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention which is defined in the claims and their equivalents.

When introducing elements of the present invention or the one or more embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

1. An elevator tool for preparing a surgical site for an implant device, the elevator tool comprising:
a paddle handle assembly;
a distal paddle;
the paddle handle assembly operably coupled to the distal paddle whereby the paddle handle assembly influences a movement of the distal paddle; and
a positioning grip extending between the paddle handle assembly and the distal paddle whereby the positioning grip influences a position of the distal paddle.

2. The elevator tool of claim 1 wherein:
the positioning grip comprises an outer sleeve;
the outer sleeve having a receiving portion; and
the paddle handle assembly is coupled to the distal paddle with an inner shaft extending through the receiving portion of the outer sleeve.

3. The elevator tool of claim 2 wherein:
the outer sleeve is coupled to a shell;
the shell having a receiving portion; and
the inner shaft extending through the receiving portion of the shell.

4. The elevator tool of claim 2 wherein:
the receiving portion of the outer sleeve is configured to allow for an extension and a retraction of the inner shaft in and out of the receiving portion of the outer sleeve;
the outer sleeve having an outer sleeve stop; and
the inner shaft has a measuring portion exposed outside of the receiving portion of the outer sleeve whereby when the outer sleeve stop is positioned against a side of a bone, the measuring portion identifies a distance from the side of the bone and the distal paddle.

5. The elevator tool of claim 1 further comprising:
the paddle handle assembly coupled to the distal paddle with an inner shaft;
a lock coupled to the positioning grip;
the paddle handle assembly comprising a handle; and
the lock configured to engage the handle whereby when the lock is engaged with the paddle handle, the handle and the distal paddle are maintained in a locked position relative to the positioning grip.

6. The elevator tool of claim 1 wherein:
the distal paddle comprises an elongated member having at least one arm extending from a rotational axis; and
the at least one arm is configured to be rotated at least about 90 degrees by the paddle handle assembly.

7. The elevator tool of claim 1 wherein:
the distal paddle comprises an elongated member having at least one arm extending from a rotational axis; and
the at least one arm is configured to be rotated at least about 180 degrees by the paddle handle assembly.

8. The elevator tool of claim 1 wherein:
the distal paddle comprises an elongated member having at least one arm extending from a rotational axis; and
the at least one arm is configured to be rotated unconstrained at least about 360 degrees by the paddle handle assembly.

9. The elevator tool of claim 1 wherein:
the positioning grip comprises an outer sleeve;
the outer sleeve having a receiving portion;
the outer sleeve coupled to a shell;
the shell having a receiving portion;
the paddle handle assembly is coupled to the distal paddle with an inner shaft extending through the receiving portion of the outer sleeve and the receiving portion of the shell;
the paddle handle assembly comprises a handle coupled to the distal paddle with an inner shaft;
the elevator tool further comprises a lock coupled to the positioning grip;
the lock configured to engage the handle whereby when the lock is engaged with the handle, the handle and the distal paddle are maintained in a locked position relative to the positioning grip;
the distal paddle comprises an elongated member having at least two arms extending from a rotational axis; and
the at least two arms are configured to be rotated at least about 180 degrees by the handle whereby the at least two arms are configured to remove obstructions on a contralateral side of a vertebral body by manipulating the handle oriented on the ipsilateral side of the vertebral body.

10. An elevator tool for use in preparing a contralateral side of a vertebral body for an orthopedic implant, the elevator tool comprising:
a paddle handle assembly;
a distal paddle;
the paddle handle assembly operably coupled to the distal paddle whereby the paddle handle assembly influences a movement of the distal paddle;
a positioning grip extending between the paddle handle assembly and the distal paddle whereby the positioning grip influences a position of the distal paddle;
the positioning grip comprises an outer sleeve;
the outer sleeve having a receiving portion;
the outer sleeve coupled to a shell;
the shell having a receiving portion;
the paddle handle assembly is coupled to the distal paddle with an inner shaft extending through the receiving portion of the outer sleeve and the receiving portion of the shell;
the paddle handle assembly comprises a handle coupled to the distal paddle with an inner shaft;
the distal paddle comprises an elongated member having at least one arm extending from a rotational axis; and
the at least one arm is configured to be rotated by the handle whereby the at least one arm is configured to remove obstructions on the contralateral side of the vertebral body by manipulating the paddle handle from a position oriented on the ipsilateral side of the vertebral body.

11. The elevator tool of claim 10 wherein:
the elevator tool further comprises a lock coupled to the positioning grip; and
the lock configured to engage the handle whereby when the lock is engaged with the handle, the handle and the distal paddle are maintained in a locked position relative to the positioning grip and when the lock is not engaged with the handle, the handle and the distal paddle are able to rotate within the receiving portion of the outer sleeve.

12. The elevator tool of claim 11 wherein the at least one arm is configured to be rotated through an unconstrained 360 degree rotation when the lock is not engaged with the handle.

13. A method for preparing a contralateral side of an insertion site of a bone for an orthopedic implant, the method comprising:
providing access to a bone;
providing an elevator tool comprising a distal paddle, a paddle handle assembly, and a shell;
the distal paddle operably coupled to the paddle handle assembly;
positioning, from an ipsilateral side of the bone, the shell in an insertion site whereby the distal paddle is positioned on a contralateral side of the bone; and
engaging the paddle handle assembly to move the distal paddle on the contralateral side of the bone to prepare the contralateral side of the bone for the orthopedic implant.

14. The method of claim 13 wherein the step of engaging the paddle handle assembly to move the distal paddle on the contralateral side of the bone clears an obstruction on the contralateral side of the bone.

15. The method of claim 13 wherein the step of engaging the paddle handle assembly to move the distal paddle on the contralateral side of the bone elevates a tissue on the contralateral side of the bone.

16. The method of claim 13 wherein the step of engaging the paddle handle assembly to move the distal paddle on the contralateral side of the bone comprises rotating the paddle handle assembly to rotate the distal paddle to clear an obstruction or elevate a tissue on the contralateral side of the bone.

17. The method of claim 13 wherein:
the paddle handle assembly is operably coupled to the distal paddle with an inner shaft rigidly coupling the paddle handle assembly to the distal paddle; and
the step of engaging the paddle handle assembly to move the distal paddle on the contralateral side of the bone comprises rotating the paddle handle assembly to rotate the inner shaft and the distal paddle whereby the distal paddle rotates to clear an obstruction or elevate a tissue on the contralateral side of the bone.

18. The method of claim 13 wherein the bone comprises a vertebral body and the contralateral side of the vertebral body comprises a contralateral sidewall of the vertebral body.

19. The method of claim 13 wherein the bone comprises a first vertebral body and a second vertebral body and the contralateral side of the bone comprises a contralateral sidewall of the first vertebral body and a contralateral sidewall of the second vertebral body.

20. The method of claim 13 wherein:
the paddle handle assembly is operably coupled to the distal paddle with a trigger mechanism; and
the step of engaging the paddle handle assembly to move the distal paddle on the contralateral side of the bone comprises engaging the trigger mechanism to rotate the distal paddle whereby the distal paddle rotates to clear an obstruction or elevate a tissue on the contralateral side of the bone.

21. The method of claim 13 wherein the step of engaging the paddle handle assembly to move the distal paddle on the contralateral side of the bone comprises engaging the paddle handle assembly to move the distal paddle in about 180 degrees of rotation on the contralateral side of the bone.

22. The method of claim 13 wherein the step of engaging the paddle handle assembly to move the distal paddle on the contralateral side of the bone comprises engaging the paddle handle assembly to move the distal paddle in an unconstrained 360 degrees of rotation on the contralateral side of the bone.

23. The method of claim 13 wherein:
the bone comprises one or more vertebra; and
the step of providing access to the one or more vertebra is from an anterior approach relative to the one or more vertebra.
